(19) United States
(12) Patent Application Publication
(10) Pub. No.: US 2026/0066120 A1
(43) Pub. Date: Mar. 5, 2026

(54) UNIFIED AI-DRIVEN CLINICAL DECISION SUPPORT AND WORKFLOW OPTIMIZATION SYSTEM

(71) Applicant: Clover Health, Jersey City, NJ (US)

(72) Inventors: Kumar Dharmarajan, New York Ciry, NY (US); Melanie Goetz, Oakland, CA (US); Chris Hartfield, San Francisco, CA (US); Jenny Korban, Chicago, IL (US); Wai Kit Kung, Tseung Kwan O, Hong Kong (CN); Jason Nance, Raleigh, NC (US); Kin Lap Tong, North Point, Hong Kong (CN); Amanda Yarmolich, Martinez, CA (US); Jimmy Yeung, Shau Kei Wan, Hong Kong (CN)

(21) Appl. No.: 19/314,749

(22) Filed: Aug. 29, 2025

Related U.S. Application Data
(60) Provisional application No. 63/689,239, filed on Aug. 30, 2024.
(60) Provisional application No. 63/689,229, filed on Aug. 30, 2024.
(60) Provisional application No. 63/689,212, filed on Aug. 30, 2024.
(60) Provisional application No. 63/689,197, filed on Aug. 30, 2024.
(60) Provisional application No. 63/689,187, filed on Aug. 30, 2024.
(60) Provisional application No. 63/689,177, filed on Aug. 30, 2024.
(60) Provisional application No. 63/689,149, filed on Aug. 30, 2024.
(60) Provisional application No. 63/689,141, filed on Aug. 30, 2024.
(60) Provisional application No. 63/689,108, filed on Aug. 30, 2024.

(51) Int. Cl. G16H 50/20; G16H 10/60; G16H 20/10; G16H 30/20; G16H 40/20; G16H 40/67; G16H 50/30; G16H 50/70; G16H 80/00

(52) U.S. Cl. CPC G16H 50/20; G16H 10/60; G16H 20/10; G16H 30/20; G16H 40/20; G16H 40/67; G16H 50/30; G16H 50/70; G16H 80/00

(57) ABSTRACT

Systems and methods for enhancing clinical decision-making and workflow optimization is proposed. An example method includes the steps of collecting data from multiple healthcare sources, including electronic health records (EHRs), medical imaging, and patient-reported outcomes. The example method also includes analyzing the collected data using artificial intelligence (AI) and machine learning (ML) algorithms to generate actionable insights. Additionally, the example method includes presenting the generated insights to healthcare providers through a unified interface.

### CROSS-REFERENCE TO RELATED APPLICATION

This Patent Application claims priority to U.S. Provisional Patent Application No. 63/689,108, filed on Aug. 30, 2024, and entitled “UNIFIED AI-DRIVEN CLINICAL DECISION SUPPORT AND WORKFLOW OPTIMIZATION SYSTE,” U.S. Provisional Patent Application No. 63/689,141, filed on Aug. 30, 2024, and entitled “CLINICAL DATA EXPLORER AND ACTIONABLE DATA PRESENTATION SYSTEM,” U.S. Provisional Patent Application No. 63/689,149, filed on Aug. 30, 2024, and entitled “INTEGRATED MEDICATION ADHERENCE AND HOSPITALIZATION MONITORING SYSTEM,” U.S. Provisional Patent Application No. 63/689,177, filed on Aug. 30, 2024, and entitled “ENHANCED HEALTHCARE AI: INTEGRATION OF LLM, NLP, AND PROPRIETARY MODELS,” U.S. Provisional Patent Application No. 63/689,187, filed on Aug. 30, 2024, and entitled “HEDIS WORKFLOW INTEGRATION WITH AUTOMATED SUSPECT HIT PROCESSING,” U.S. Provisional Patent Application No. 63/689,197, filed on Aug. 30, 2024, and entitled “AI-DRIVEN TREATMENT RECOMMENDATION AND CONTRAINDICATION IDENTIFICATION SYSTEM,” U.S. Provisional Patent Application No. 63/689,212, filed on Aug. 30, 2024, and entitled “TASK TRIAGE WORKFLOW OPTIMIZATION SYSTEM FOR DISEASE MANAGEMENT,” U.S. Provisional Patent Application No. 63/689,229, filed on Aug. 30, 2024, and entitled “ENSEMBLE MACHINE LEARNING MODEL FRAMEWORK FOR CLINICAL DECISION SUPPORT,” and U.S. Provisional Patent Application No. 63/689,239, filed on Aug. 30, 2024, and entitled “POPULATION HEALTH METRICS AND MEDICATION ADHERENCE TRACKING TOOL.” The disclosures of the prior Applications are considered part of and are incorporated by reference into this Patent Application.

### TECHNICAL FIELD

The present disclosure relates generally to healthcare information technology and, more specifically, to healthcare data systems.

### BACKGROUND

The integration of advanced technologies in healthcare has significantly transformed the industry, enabling more efficient management of patient data and clinical workflows. Healthcare providers increasingly rely on data-driven insights and automation to enhance patient care, streamline operations, and reduce costs. The rapid advancements in artificial intelligence (AI), machine learning (ML), and natural language processing (NLP) has the potential to open new avenues for improving healthcare outcomes by providing more accurate diagnostics, personalized treatment recommendations, and proactive health management.

However, the healthcare industry faces challenges in fully leveraging these technologies. Issues such as data fragmentation, the complexity of integrating diverse data sources, and the need for real-time decision support are common. Moreover, there is an increasing interest for systems that can handle vast amounts of clinical data, ensure regulatory compliance, and maintain patient privacy.

To address these challenges, there is a need for comprehensive systems that can integrate AI, ML, and NLP technologies to enhance clinical decision-making, automate routine tasks, and provide actionable insights. Such systems can support healthcare providers in delivering higher-quality care, improving patient outcomes, and optimizing healthcare operations. The present disclosure addresses these needs by providing a unified approach to healthcare information technology, encompassing various aspects such as clinical data exploration, medication adherence monitoring, workflow optimization, and advanced analytics.

### SUMMARY

The following presents a simplified summary of one or more aspects in order to provide a basic understanding of such aspects. This summary is not an extensive overview of all contemplated aspects and is intended to neither identify key or critical elements of all aspects nor delineate the scope of any or all aspects. Its sole purpose is to present some concepts of one or more aspects in a simplified form as a prelude to the more detailed description that is presented later.

An example method includes the steps of collecting data from multiple healthcare sources, including electronic health records (EHRs), medical imaging, and patient-reported outcomes. The example method also includes analyzing the collected data using artificial intelligence (AI) and machine learning (ML) algorithms to generate actionable insights. Additionally, the example method includes presenting the generated insights to healthcare providers through a unified interface.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a diagram illustrating a clinical data explorer and actionable data presentation system in accordance with the systems and methods described herein.

FIG. 2 is a diagram illustrating a screen for reviewing suspected diagnoses in accordance with the systems and methods described herein.

FIG. 3 is a diagram illustrating an assistant screen that may be used in an integrated medication adherence and hospitalization monitoring system in accordance with the systems and methods described herein.

FIG. 4 is a diagram illustrating an assistant screen for a flagged items in accordance with the systems and methods described herein.

FIG. 5 is a diagram illustrating an assistant screen for a particular disease in accordance with the systems and methods described herein.

FIGS. 6A-6B illustrate a flow diagram for an example method of AI-driven collection, integration, and analysis of healthcare data from multiple sources to provide unified insights, automate tasks, and support clinical decision-making in accordance with the systems and methods described herein.

FIGS. 7A-7B illustrate a flow diagram for an example method of NLP-powered extraction, organization, and presentation of key information from clinical documents with search, visualization, and alert features in accordance with the systems and methods described herein.

FIGS. 8A-8B illustrate a flow diagram for an example method of monitoring medication adherence and hospitalization events to generate reports, provide alerts, and support interventions in accordance with the systems and methods described herein.

FIGS. 9A-9B illustrate a flow diagram for an example method of using large language models with NLP to extract, translate, and summarize clinical information for multilingual, accessible decision support in accordance with the systems and methods described herein.

FIGS. 10A-10B illustrate a flow diagram for an example method of detecting, prioritizing, and reviewing quality-measure “suspect hits” for HEDIS compliance, with reporting and integration into quality workflows in accordance with the systems and methods described herein.

FIGS. 11A-11B illustrate a flow diagram for an example method of AI analysis of patient data to generate personalized treatment recommendations, identify contraindications, and provide actionable explanations in accordance with the systems and methods described herein.

FIGS. 12A-12B illustrate a flow diagram for an example method of AI-driven clinical task management that categorizes, prioritizes, assigns, and tracks tasks, integrating with EHRs and supporting analytics in accordance with the systems and methods described herein.

FIGS. 13A-13B illustrate a flow diagram for an example method to ensemble machine learning analysis combining multiple specialized models to generate predictions, insights, and decision support alerts in accordance with the systems and methods described herein.

FIGS. 14A-14B illustrate a flow diagram for an example method of population-level health monitoring with adherence tracking, risk stratification, alerts, and personalized interventions in accordance with the systems and methods described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

The following detailed description is intended to provide example embodiments of the claimed methods and systems. Unless explicitly stated otherwise, no statement herein should be construed as limiting the scope of the claims to a specific embodiment. Various elements described may be substituted, combined, or rearranged in different examples.

The detailed description set forth below in connection with the appended drawings is intended as a description of configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

The described methods may be implemented using one or more computing platforms that may include server-class machines, cloud computing environments, distributed edge computing devices, or combinations thereof. Hardware may include processors (e.g., CPUs, GPUs, TPUs, FPGAs), system memory (e.g., DRAM, SRAM), storage devices (e.g., SSDs, HDDs, RAID arrays), network interface controllers for wired and wireless connections, display devices (e.g., monitors, tablets), and input devices (e.g., keyboards, mice, touchscreens, microphones). The systems may communicate with healthcare data sources such as electronic health record (EHR) systems, laboratory information systems, radiology imaging systems, pharmacy dispensing networks, wearable medical devices, and remote patient monitoring platforms. Data communication may use secure APIs, HL7/FHIR interfaces, VPN tunnels, or other secure networking protocols.

### Enhancing Clinical Decision-Making and Workflow Optimization

In some examples, a method may collect data from multiple healthcare sources. These may include EHR systems with structured records (e.g., demographic data, vital signs, laboratory results) and unstructured data (e.g., clinical notes, operative reports). Other sources may include medical imaging repositories storing radiology, cardiology, or pathology images in DICOM format; patient-reported outcomes from web surveys, kiosks, or mobile health apps; and biosensor data from wearable devices such as heart rate monitors or continuous glucose monitors.

The collected data may be processed using artificial intelligence (AI) and machine learning (ML) models. These may include supervised learning classifiers for disease prediction, unsupervised clustering models for patient segmentation, deep learning convolutional networks for image analysis, and reinforcement learning agents for treatment optimization.

Generated insights may be presented via a unified interface. This interface may be a responsive web dashboard, a native mobile application, or an embedded module within an existing EHR system. Insights may be displayed as tables, color-coded risk indicators, timelines, or interactive patient journey maps.

Routine administrative tasks may be automated, including appointment scheduling via calendar integrations, billing and coding through automated claim generation, and documentation using auto-scribing systems that transcribe spoken conversations into structured notes. Decision support alerts may flag potential drug-drug interactions, allergies, or predicted high-risk patient events.

The system may ensure interoperability with existing healthcare systems by adhering to HL7 FHIR, DICOM, IHE profiles, or other data exchange standards. AI/ML models may be continuously updated as new data is collected, ensuring that recommendations remain current. Genetic data and lifestyle factors, such as dietary habits, activity levels, and environmental exposures, may also be incorporated to refine personalization. Collaboration features may include shared patient records, secure chat, and telehealth video integrations. Security measures may include AES-256 encryption, role-based permissions, and detailed audit logs.

### Exploring and Presenting Clinical Data

A method may receive clinical documents from various sources, including hospital EHR exports, scanned paper records processed through OCR, or HL7 message feeds. These documents may include unstructured free-text clinical notes, operative reports, pathology reports, referral letters, discharge summaries, and structured forms.

Natural language processing (NLP) algorithms may parse and extract relevant information such as diagnoses, treatment plans, medication history, lab results, and clinical observations. Extracted data points may be highlighted in the user interface for rapid review, and abnormal values may be flagged for attention.

The information may be organized into actionable formats such as patient summaries, chronological care timelines, or condition-specific overviews. Search functionality may allow users to query specific terms, ICD-10 codes, or drug names. Tagging and categorization may be automated using AI models trained on medical ontologies, such as SNOMED CT or RxNorm.

Visualization tools may include bar charts of lab trends, medication timelines, or network graphs showing relationships among conditions and treatments. Users may annotate and add notes to highlighted data points. Multilingual support may enable use in diverse patient populations, with real-time translation of medical terms.

### Monitoring Medication Adherence and Hospitalization Events

This method may collect adherence data from pharmacy refill records, pill bottle smart caps, mobile self-report surveys, and EHR prescription logs. Adherence patterns may be tracked by identifying missed refills, late pickups, or self-reported skipped doses.

Hospitalization events may be monitored through EHR admission/discharge notifications, insurance claim submissions, or patient self-reporting. Data may include admission reason, length of stay, discharge instructions, and follow-up requirements.

Machine learning models may predict risk of future hospitalizations based on adherence history, comorbidities, and demographic risk factors. Personalized interventions may include reminder notifications, pharmacist outreach, or medication synchronization programs.

Reports may summarize adherence percentages, hospitalization frequency, and risk trends. Secure patient portals may allow individuals to view their own data and communicate with providers.

### Enhancing Healthcare Data Analysis Through AI Integration

In some embodiments, large language models (LLMs) may be integrated with NLP pipelines to interpret clinical notes, patient histories, and other unstructured data. Proprietary models may be optimized for specific specialties such as oncology, cardiology, or endocrinology.

The system may translate complex medical terms into patient-friendly plain language, supporting provider-patient communication. Summarized insights may be generated, highlighting key findings such as diagnosis progression or treatment outcomes.

Multilingual processing may support translation into languages such as Spanish, Mandarin, or Arabic. Feedback mechanisms may allow providers to flag incorrect interpretations, retraining the models over time.

### Automating Suspect Hit Processing in HEDIS Workflows

This method may identify potential quality measure gaps (suspect hits) by analyzing claims, EHR data, and lab results. Predefined rules and ML algorithms may determine if a patient is missing a measure such as a cancer screening, immunization, or lab test.

Suspect hits may be prioritized based on measure impact, patient risk, or regulatory deadlines. Confirmation of a primary hit may deprioritize related hits to reduce duplication. Explanations may cite relevant guidelines (e.g., NCQA HEDIS specifications) and link to supporting patient data.

Reports may track the review process, generating audit trails. Alerts may notify providers of new suspect hits, and secure communication tools may facilitate discussion with quality teams.

### AI-Driven Treatment Recommendations and Contraindication Identification

This method may analyze patient history, genetic data, current medications, and diagnostic results to propose treatment options. Contraindications may be identified from known drug interactions, allergies, or coexisting conditions.

Recommendations may be displayed in a structured interface with supporting evidence, risk/benefit summaries, and alternative options if contraindications are present. Explanations may include visual risk scores or treatment timelines.

Integration with EHRs may allow seamless ordering of recommended treatments. Multilingual support may assist providers working with diverse populations.

### Optimizing Task Triage Workflows in Disease Management

Tasks may be categorized by urgency (critical, high, normal, low) and importance using AI models trained on historical workflow data. High-priority tasks may include urgent follow-up appointments, imaging requests, or abnormal lab result reviews.

The system may allocate tasks to available providers based on specialty, workload, or location. Alerts and reminders may be delivered via email, SMS, or in-app notifications. Collaboration tools may enable shared task boards and progress tracking.

Analytics modules may track task completion rates, delays, and bottlenecks for process improvement.

### Clinical Decision Support Using Ensemble Machine Learning Models

Multiple specialized ML models may be integrated into an ensemble framework, with each model focused on specific tasks such as image analysis, lab trend prediction, or treatment response forecasting. A combination algorithm may merge outputs to produce consensus recommendations.

The framework may handle conflicting outputs by weighting models based on accuracy history. Explanations may be provided using visual aids like annotated images or correlation graphs. EHR integration may allow results to be presented within provider workflows. Models may be retrained as new research and data become available.

### Tracking Population Health Metrics and Medication Adherence

Population health data may include prevalence of chronic conditions (e.g., diabetes, COPD), risk factors (e.g., obesity rates, smoking prevalence), and healthcare utilization statistics. Medication adherence tracking may use aggregated pharmacy data, patient-reported adherence, and EHR prescribing records.

Patients may be stratified into risk tiers for targeted outreach. Alerts may be generated for those with critical adherence issues. Educational resources may be delivered via patient portals or mobile apps.

Predictive analytics may identify individuals at risk of hospitalization, allowing preemptive intervention.

The present disclosure provides a comprehensive healthcare information technology system that may integrate one or more of artificial intelligence (AI), machine learning (ML), and natural language processing (NLP) to enhance clinical decision-making, workflow optimization, and patient management. The system encompasses various components and functionalities designed to address key challenges in healthcare, as detailed herein.

### Unified AI-Driven Clinical Decision Support and Workflow Optimization System

This comprehensive system aims to integrate various AI and ML technologies into a unified platform that supports clinical decision-making and workflow optimization. In some example embodiments, the system may collect and analyzes data from multiple sources, including electronic health records (EHRs), medical imaging, and patient-reported outcomes, to generate actionable insights. By leveraging advanced analytics, the system helps healthcare providers make more informed decisions, improving patient outcomes and operational efficiency.

In some example embodiments, the workflow optimization aspect of the system may include tools for automating routine tasks, such as appointment scheduling, billing, and documentation. This may reduce administrative burdens on healthcare staff and allows them to focus more on patient care. The system may also includes decision support features that alert providers to potential issues, such as drug interactions or contraindications, ensuring safer and more effective treatment plans.

Some example embodiments may include integration with existing healthcare systems is a key feature, enabling seamless data exchange and interoperability. An example system may support various healthcare standards and protocols, ensuring compatibility with a wide range of clinical software and devices. This flexibility may make the system suitable for diverse healthcare settings, from small clinics to large hospitals.

In some example embodiments, some example methods for this system involves several steps, starting with the integration of data from multiple healthcare sources. This may include EHRs, medical imaging, and patient-reported outcomes, each of which is aggregated and standardized to ensure compatibility. The system may employ advanced AI and ML algorithms to process this data, extracting relevant clinical insights and predicting potential patient outcomes. These insights may not only be generated but also contextualized to reflect the patient's unique medical history and current health status.

In some example embodiments, the system presents these insights through a sophisticated, intuitive user interface that allows healthcare providers to access information swiftly. The system may organize data into easily navigable sections, highlighting key metrics such as lab results, imaging findings, and medication histories. The system may automate routine administrative tasks, such as scheduling follow-up appointments and processing billing codes, which may be streamlined using AI-driven workflows. These workflows may be designed to minimize manual entry and reduce the potential for human error, enhancing overall efficiency.

In some example embodiments, a significant component of this system may be the system's decision-support capabilities. The system may generate alerts for potential issues, such as adverse drug interactions, based on real-time analysis of the patient's medications and medical history. The system may also suggest best practices and treatment guidelines tailored to individual patient cases, supported by the latest medical research and guidelines. The AI and ML models may be continuously refined and updated with new data, including genomic information and lifestyle factors, ensuring that the insights provided may be current and relevant.

In some example embodiments, the system may be designed to be interoperable with existing healthcare infrastructure, adhering to standard healthcare data protocols and formats. This may ensure seamless data exchange between different systems and devices, facilitating a cohesive healthcare ecosystem. Security and compliance may be important in some aspects. In such a system, the system may employ robust encryption and access control mechanisms to protect sensitive patient information, ensuring compliance with regulations such as HIPAA.

### Clinical Data Explorer and Actionable Data Presentation System

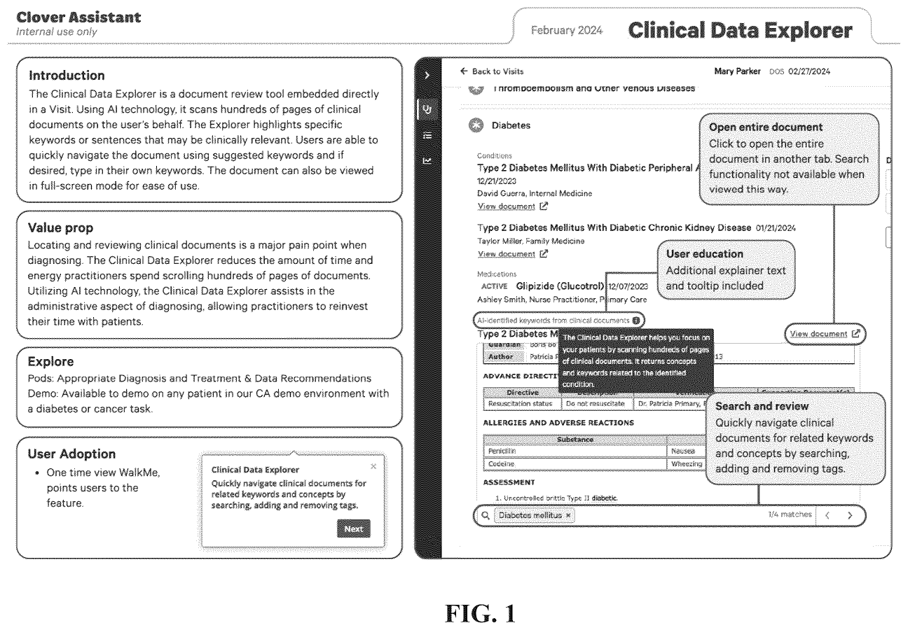

FIG. 1 is a diagram illustrating a Clinical Data Explorer and Actionable Data Presentation System in accordance with the systems and methods described herein. The Clinical Data Explorer may, in some example embodiments, be designed to may simplify the review and analysis of large volumes of clinical data. The system may utilize AI and NLP to scan documents, extract relevant information, and present the information in an organized, easy-to-navigate format. This tool may be particularly useful for handling unstructured data, such as free-text clinical notes, which may often be challenging to analyze systematically.

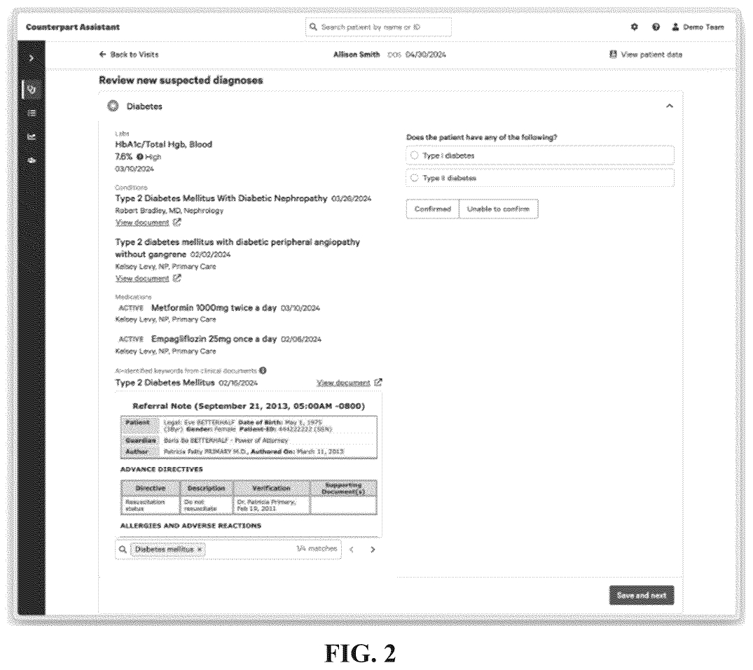

FIG. 2 is a diagram illustrating a screen for reviewing suspected diagnoses in accordance with the systems and methods described herein. In some example embodiments, by highlighting key information, such as diagnoses, treatment plans, and medication history, the system allows healthcare providers to quickly identify details. The tool may include a search function that supports both keyword and concept-based queries, enabling users to find specific information efficiently. The ability to tag and categorize data may further enhance the system's usability, making it easier to manage and reference important clinical information.

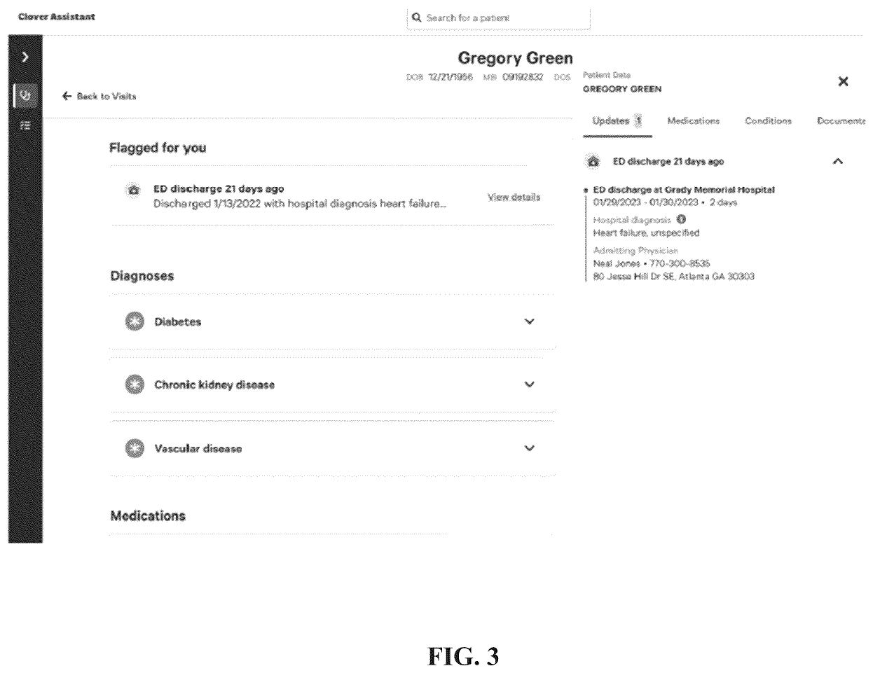

FIG. 3 is a diagram illustrating an assistant screen that may be used in an integrated medication adherence and hospitalization monitoring system in accordance with the systems and methods described herein. In some example embodiments, the actionable data presentation aspect may ensure that the extracted information is not only accessible but also useful for clinical decision-making. The system may flag abnormal values, suggest possible diagnoses, and provide summaries of patient histories. This functionality may help providers quickly assess patient conditions and make informed decisions, ultimately improving the quality of care.

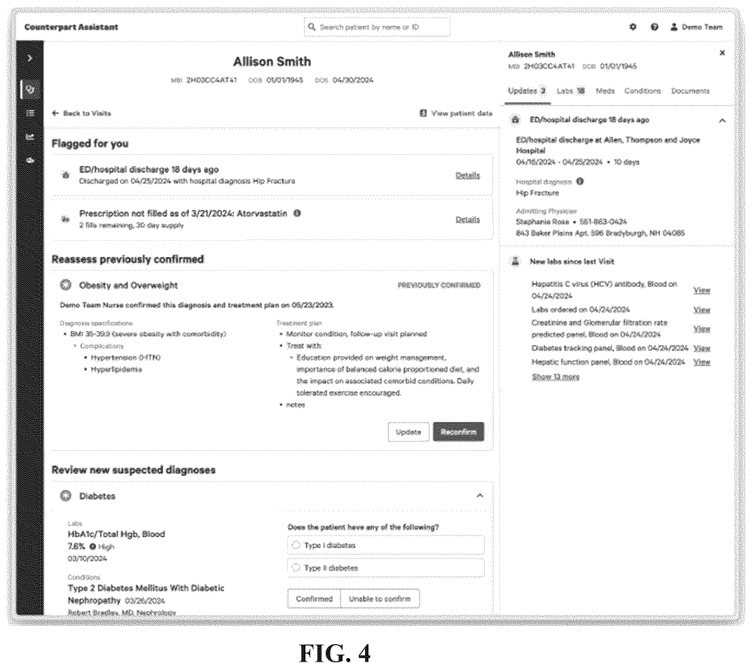

FIG. 4 is a diagram illustrating an assistant screen for a flagged items in accordance with the systems and methods described herein. In some example embodiments, this method involves a multi-step process that begins with the ingestion of diverse clinical documents from various healthcare entities. These documents often include complex, unstructured data, such as detailed clinical notes, imaging reports, and patient histories. The system may leverage sophisticated NLP algorithms to parse these documents, identifying and extracting key clinical information. This process may involve recognizing medical terminology, contextualizing findings, and correlating them with patient-specific data.

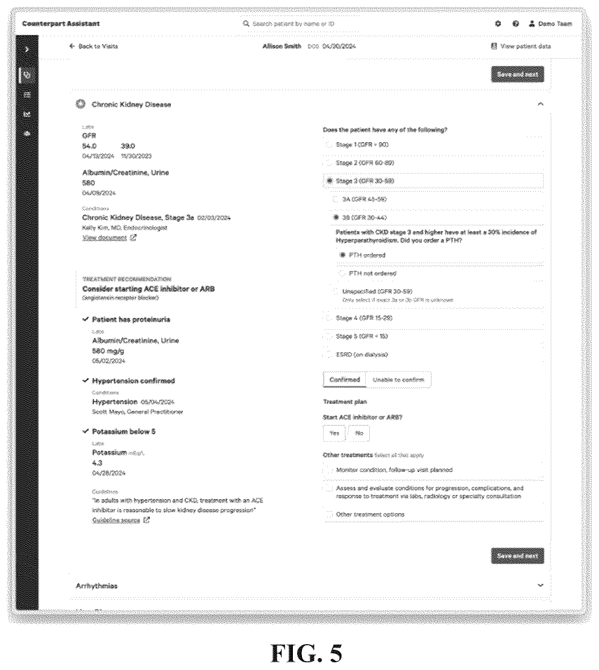

FIG. 5 is a diagram illustrating an assistant screen for a particular disease in accordance with the systems and methods described herein. In some example embodiments, the extracted information may then be organized into a coherent, actionable format, which is presented through a highly interactive user interface. The system may allow healthcare providers to conduct in-depth searches using both keywords and more complex queries, which can be refined by context or specific clinical parameters. This search capability may be augmented by tagging and categorization features, which organize information according to relevance and clinical priority.

In some example embodiments, to help ensure that data points are not overlooked, the system may automatically flag abnormal values and significant findings, such as unusual lab results or deviations from expected clinical progress. These flagged items may be prioritized and highlighted, prompting immediate attention from healthcare providers. Visualization tools may be integrated into the system, providing graphical representations of data trends, such as changes in vital signs over time or progression of disease markers, which aid in the clinical decision-making process.

In some example embodiments, the system supports multilingual data presentation, making the data accessible to a wide range of users and accommodating patients who speak different languages. Additionally, the system may include tools for annotation and note-taking, allowing healthcare providers to add contextual information or personal insights directly to the clinical data. This capability may enhance the collaborative aspect of patient care, as multiple providers can share insights and updates in real-time, near real-time, or substantially real-time.

### Integrated Medication Adherence and Hospitalization Monitoring System

In some example embodiments, this system integrates data from various healthcare sources to provide a comprehensive view of a patient's medication adherence and hospitalization history. The system may monitor adherence to prescribed medications by analyzing pharmacy data, patient self-reports, and electronic health records. In some example embodiments, the system may detect patterns of non-adherence, such as missed doses or irregular medication intake, which may be needed for managing chronic conditions.

In some example embodiments, the hospitalization monitoring component tracks admissions and discharges, providing details such as the reason for hospitalization, length of stay, and follow-up care. This information may be needed for understanding a patient's overall health status and identifying potential risk factors. The system may alert healthcare providers to recent hospitalizations, enabling timely follow-up and intervention.

In some example embodiments, by combining medication adherence and hospitalization data, the system offers valuable insights into patient health behaviors and outcomes. An example system may identify patients at high risk of adverse events, such as readmissions or complications, and suggest targeted interventions. This comprehensive monitoring capability may support proactive healthcare management, helping to reduce costs and improve patient outcomes.

In some example embodiments, this method starts with the integration of data from various healthcare sources, such as pharmacy records, patient self-reports, and electronic health records (EHRs). The system may be designed to track medication adherence by analyzing these data sources to identify patterns of non-adherence, such as missed doses or irregular medication intake. An example system may utilize sophisticated algorithms to correlate medication adherence with clinical outcomes, providing a comprehensive view of a patient's medication management.

In some example embodiments, simultaneously, the system monitors hospitalization events, including admissions, discharges, and readmissions. An example system may capture detailed information about each hospitalization, such as the cause, duration, treatments received, and any follow-up care requirements. This data may be synthesized into a unified patient profile that provides healthcare providers with a complete overview of the patient's health status and medical history.

In some example embodiments, healthcare providers may access this comprehensive profile through an intuitive dashboard that highlights key areas of concern, such as frequent hospitalizations or persistent medication non-adherence. An example system may generate alerts for healthcare providers, notifying them of issues that require immediate attention, such as a recent discharge without a scheduled follow-up. These alerts may be based on real-time, near real-time, or substantially real-time data analysis and may be designed to prompt timely interventions that can prevent adverse events, such as medication errors or preventable readmissions.

In some example embodiments, to enhance patient engagement, the system provides patients with access to their own medication and hospitalization records through a secure patient portal. This portal may include features such as medication reminders, adherence tracking, and educational resources, empowering patients to take an active role in their healthcare. The system may also ensure compliance with healthcare regulations by implementing stringent security measures to protect patient data, including data encryption and secure authentication protocols, in some example embodiments.

### Enhanced Healthcare AI: Integration of LLM, NLP, and Proprietary Models

In some example embodiments, this component may integrate large language models (LLMs) with off-the-shelf NLP technologies and proprietary models to enhance the understanding and processing of clinical data. LLMs may be advanced AI models capable of understanding and generating human language, making them ideal for interpreting complex medical information. In some example embodiments, the system uses these models to process unstructured data, such as clinical notes and patient histories, extracting relevant details and summarizing key points.

In some example embodiments, the integration of NLP technologies allows the system to handle specific medical terminologies and context-sensitive information, improving the accuracy of data extraction. In some example embodiments, proprietary models, tailored to specific healthcare applications, further enhance the system's capabilities. These models may identify subtle patterns and correlations in the data, providing deeper insights into patient conditions and treatment outcomes.

In some example embodiments, one of the features of this integration may be the ability to translate complex medical language into plain text. This functionality may help bridge the communication gap between healthcare providers and patients, ensuring that all stakeholders have a clear understanding of medical information. The system may also support multilingual capabilities, accommodating patients and providers from diverse linguistic backgrounds.

In some example embodiments, the method involves the integration of advanced large language models (LLM) with NLP technologies and proprietary models specifically designed for healthcare applications. The system may process complex clinical data, including unstructured text such as detailed clinical notes and patient histories. Using these integrated models, the system may extract and interpret complex medical information, converting the medical information into easily understandable summaries.

In some example embodiments, the system's ability to understand and generate human-like language may be enhanced by the system's contextual awareness, which allows the system to distinguish between different uses of similar terms based on the medical context. This capability may be particularly valuable in differentiating between nuanced medical conditions and treatment protocols. The proprietary models may complement the LLM and NLP components by offering specialized algorithms that may be fine-tuned for specific tasks, such as diagnosing rare diseases or predicting treatment outcomes.

In some example embodiments, in addition to generating plain language summaries, the system supports multilingual capabilities, enabling the system to process and translate medical information into multiple languages. This feature may be used for providing care to diverse patient populations and ensuring that all patients receive comprehensible and accurate medical information. The system also provides detailed explanations of the system's analyses, including the reasoning behind certain medical insights and recommendations, which may be presented in a format accessible to both healthcare providers and patients in some example embodiments.

In some example embodiments, to maintain high accuracy and relevance, the system may be continuously updated with the latest medical research and clinical data. Feedback loops may allow healthcare providers to correct or refine the system's outputs, which helps improve system performance over time. Data privacy and security may be prioritized, with robust measures in place to safeguard sensitive medical information and comply with healthcare regulations.

### HEDIS Workflow Integration With Automated Suspect Hit Processing

In some example embodiments, the system automates the process of generating and reviewing suspect hits within the HEDIS workflow, a set of standardized performance measures used to assess the quality of care in healthcare settings. Suspect hits may be potential issues identified in patient records that may require further investigation or confirmation. The system may prioritize these hits based on their relevance and accuracy, streamlining the review process.

By automating the generation of suspect hits, the system may reduce the manual effort required to identify quality concerns in some examples. The system may use AI algorithms to analyze patient data, flagging potential issues such as gaps in care, missed screenings, or incomplete documentation. Once a suspect hit may be confirmed, related hits may be deprioritized, minimizing redundant work and focusing attention on the most critical issues.

In some example embodiments, the integration improves the efficiency and effectiveness of quality assessment in healthcare. An example system may ensure that providers are alerted to potential issues in a timely manner, allowing for prompt corrective actions. In some example embodiments, the system may also supports comprehensive reporting and auditing capabilities, helping organizations maintain compliance with regulatory requirements and quality standards.

In some example embodiments, this method automates the process of generating and managing suspect hits within the Healthcare Effectiveness Data and Information Set (HEDIS) workflow. The system may start by analyzing patient data to identify potential quality measures that may indicate gaps in care or areas requiring further evaluation. These potential issues, known as suspect hits, may generated based on a set of predefined criteria and advanced algorithms that consider various patient data points in some example embodiments.

In some example embodiments, once generated, suspect hits may be prioritized based on their relevance and potential impact on patient care quality. The system may present these prioritized hits to healthcare providers, who can review and confirm their accuracy. If a suspect hit may be confirmed as relevant, the system automatically deprioritizes related hits, reducing redundancy and focusing attention on the most critical issues. This prioritization process may be supported by detailed explanations and supporting evidence, which help healthcare providers understand the context and rationale behind each hit in some example embodiments.

In some example embodiments, healthcare providers may interact with the system through an interface that allows for annotation, comments, and status updates on each suspect hit. The system may maintain an audit trail, documenting the review and decision-making process, which may be for accountability and transparency in quality assurance activities. Real-time, near real-time, or substantially real-time alerts notify providers of new suspect hits as they may be identified, ensuring timely intervention in some example embodiments.

In some example embodiments, the system also includes training and educational resources to help providers understand how to address and resolve suspect hits. These resources may include best practice guidelines, clinical pathways, and examples of how to handle specific types of quality measures. Security features protect patient data throughout this process, ensuring compliance with healthcare regulations and maintaining the confidentiality of patient information in some example embodiments.

### AI-Driven Treatment Recommendation and Contraindication Identification System

In some example embodiments, this system leverages AI and ML models to provide personalized treatment recommendations and identify contraindications. An example system may analyze patient data, including medical history, genetic information, and current medications, to suggest appropriate treatment options. The system may also highlight potential contraindications, such as drug interactions or medical conditions that may preclude certain treatments in some example embodiments.

In some example embodiments, the recommendation engine may use advanced algorithms to evaluate the efficacy and safety of various treatment options. An example system may consider a wide range of factors, including clinical guidelines, patient preferences, and emerging medical research. The system may provide a rationale for each recommendation, translating complex model outputs into understandable plain text. This transparency helps healthcare providers make informed decisions and communicate effectively with patients in some example embodiments.

In some example embodiments, in addition to treatment recommendations, the system may offer tools for monitoring and managing ongoing treatments. An example system may track patient responses, adjust treatment plans as needed, and alert providers to potential issues. This comprehensive approach may support continuous care management, ensuring that patients receive the most effective and appropriate treatments over time in some example embodiments.

In some example embodiments, the method for providing AI-driven treatment recommendations involves a detailed analysis of patient data, including medical history, genetic information, and current medications. The system may employ AI algorithms to evaluate this data, considering factors such as patient demographics, past treatments, and known drug interactions. Based on this comprehensive analysis, the system may generate personalized treatment recommendations that may be tailored to the individual patient's needs.

In some example embodiments, in addition to recommending treatments, the system identifies potential contraindications, such as adverse drug interactions or medical conditions that may contraindicate certain therapies. These contraindications may be highlighted and explained in detail, with the system providing the reasoning behind each alert. This information may be needed for healthcare providers as they assess the suitability of recommended treatments for their patients.

In some example embodiments, the recommendations and contraindications may be presented through a user-friendly interface that allows healthcare providers to review and adjust the proposed treatment plans. Providers may input additional patient-specific data or preferences, which the system incorporates into the recommendations, ensuring a highly personalized approach to care. The system may also support real-time, near real-time, or substantially real-time updates, allowing healthcare providers to modify treatment plans as new data becomes available or as the patient's condition evolves.

In some example embodiments, the system includes multilingual support, ensuring that recommendations and explanations may be accessible to providers and patients who speak different languages. Visual aids, such as charts and graphs, may be used to illustrate the relationships between various data points, making complex medical information more understandable. The system may help ensure data privacy and security, with robust measures in place to protect patient information and comply with regulatory requirements in some example embodiments.

### Task Triage Workflow Optimization System for Disease Management

In some example embodiments, the task triage workflow optimization system may be designed to prioritize and manage tasks related to disease management. An example system categorizes tasks based on urgency and importance, helping healthcare providers focus on important issues. The system uses AI to analyze patient data, identifying key tasks such as follow-up appointments, diagnostic tests, or treatment adjustments that require immediate attention.

In some example embodiments, the system includes a task management interface that allows providers to track and complete tasks efficiently. The system may provide alerts and reminders for upcoming tasks, ensuring that nothing is overlooked. The system also supports collaboration among healthcare teams, enabling providers to assign tasks, share updates, and coordinate care effectively.

In some example embodiments, by optimizing task triage workflows, the system reduces the administrative burden on healthcare providers and enhances the quality of care. An example system may help ensure that important tasks may be addressed promptly, improving patient outcomes and operational efficiency. The system also supports data-driven decision-making, providing insights into task completion rates, bottlenecks, and areas for improvement.

In some example embodiments, this method focuses on optimizing the triage of clinical tasks within the context of disease management. The system categorizes tasks based on their urgency and importance, using AI algorithms that analyze patient data to prioritize actions that require immediate attention. This prioritization takes into account factors such as the severity of the patient's condition, the potential for deterioration, and the availability of resources.

In some example embodiments, once tasks are prioritized, the system allocates them to the appropriate healthcare providers based on their expertise and current workload. This allocation may be managed through a task management interface that provides a clear overview of all pending tasks, along with their respective priorities and deadlines. The interface supports real-time, near real-time, or substantially real-time updates, allowing providers to track the status of each task, receive reminders, and adjust priorities as necessary.

In some example embodiments, the system facilitates collaboration among healthcare teams by enabling the sharing of task assignments and updates. Providers can communicate directly within the platform, share relevant patient information, and coordinate care more effectively. This collaborative approach may be particularly important in complex cases where multidisciplinary care may be required.

In some example embodiments, in addition to managing current tasks, the system provides analytics and reporting features that offer insights into workflow efficiency. These reports help identify bottlenecks and areas where improvements can be made, such as reallocating resources or streamlining specific processes. The system also allows for manual adjustments, enabling healthcare providers to reprioritize tasks or reassign them based on changing clinical circumstances.

### Ensemble Machine Learning Model Framework for Clinical Decision Support

In some example embodiments, the ensemble machine learning model framework combines multiple ML models to provide robust clinical decision support. Each model in the ensemble may be specialized in a different aspect of patient care, such as diagnosing specific conditions, predicting treatment outcomes, or assessing risk factors. By integrating these models, the framework offers comprehensive insights that consider various dimensions of patient health.

In some example embodiments, the ensemble approach enhances the accuracy and reliability of predictions and recommendations. An example system may leverage the strengths of each model, compensating for individual weaknesses and providing a more balanced assessment. The framework includes mechanisms for model validation and calibration, helping ensure that the outputs are consistent and accurate in some example embodiments.

In some example embodiments, this system may be particularly valuable in complex clinical scenarios where multiple factors may be considered. For example, in cases with co-morbid conditions or conflicting treatment options, the ensemble framework can provide a nuanced analysis that helps healthcare providers navigate difficult decisions. The system's ability to integrate diverse data sources and analytical perspectives makes these systems and methods a powerful tool for comprehensive clinical decision support.

In some example embodiments, the method involves the integration of multiple machine learning models into an ensemble framework, each model specializing in different aspects of patient care. These models analyze a comprehensive range of patient data, including clinical records, imaging results, laboratory data, and genetic information. The ensemble framework synthesizes the outputs from these models, providing a holistic view of the patient's health status and potential treatment pathways.

In some example embodiments, the system presents these integrated insights through a unified interface, where healthcare providers can explore detailed explanations and justifications for each recommendation. The ensemble approach ensures that the strengths of individual models may be combined, enhancing the accuracy and reliability of the overall predictions. For instance, one model may specialize in predicting cardiovascular risks, while another focuses on potential responses to cancer treatments; together, they provide a more complete picture of the patient's health.

In some example embodiments, providers can interact with the system by inputting additional data or specifying particular concerns, which the system then incorporates into the analysis. The ensemble framework includes mechanisms for resolving conflicting outputs from different models, generating a consensus recommendation that considers all relevant factors. This capability may be used for complex cases where multiple treatment options may be available, each with its own set of risks and benefits, for example. Other cases may also be possible.

In some example embodiments, the system may be continuously updated with new clinical data and research findings, ensuring that the models remain up-to-date and relevant. Data privacy and security measures may be rigorously applied to protect patient information, including encryption and secure access protocols. The system's compliance with healthcare regulations may be continually monitored and enforced, ensuring that all data handling processes meet the necessary legal and ethical standards.

### Silver Standard Machine Learning Models for Diagnostic Suspecting

In some example embodiments, the silver standard machine learning models may be designed to provide reliable diagnostic suspecting capabilities. These models may be trained on high-quality, well-validated data sets, ensuring that their outputs may be accurate and dependable. The term “silver standard” reflects the high level of rigor and reliability associated with these models, positioning them as a trusted resource for healthcare providers.

In some example embodiments, the diagnostic suspecting system uses these models to analyze patient symptoms, medical history, and other relevant data. An example system may generate potential diagnoses, ranking them based on likelihood and relevance. The system also provides explanations for each suspected diagnosis, helping providers understand the underlying reasoning and make informed decisions.

In some example embodiments, to ensure ongoing accuracy, the silver standard models may be continuously updated and validated against new data. This process involves cross-validation, sensitivity analysis, and other techniques to verify model performance. The system's commitment to maintaining high standards of reliability may make the system a valuable tool for supporting accurate and timely diagnoses in clinical practice.

In some example embodiments, this method focuses on the development and deployment of silver standard machine learning models, specifically designed for diagnostic suspecting. These models may be trained on high-quality, well-validated datasets to ensure that their diagnostic outputs may be accurate and reliable. The system analyzes a variety of patient data, including symptoms, medical history, diagnostic test results, and imaging findings, to generate a ranked list of potential diagnoses.

In some example embodiments, the potential diagnoses may be presented with detailed explanations, outlining the reasoning and data points that led to each suspicion. This transparency allows healthcare providers to understand the system's decision-making process, which may be needed for trust and adoption. The system also highlights the likelihood of each diagnosis, helping providers prioritize further testing or treatment based on the most probable conditions.

In some example embodiments, healthcare providers can review these suspected diagnoses and confirm, modify, or dismiss them as appropriate. The system includes tools for providers to input additional observations or clinical notes, which may then be used to refine the model's outputs. This interactive process helps improve the accuracy of the models over time, as they learn from real-world clinical decisions.

In some example embodiments, the system may be designed to generate alerts for diagnoses, such as, in some examples, critical or urgent diagnoses, prompting immediate clinical action when necessary. Some example embodiments also include features for secure storage and retrieval of patient data, ensuring that all diagnostic information may be handled with the highest level of security. The system supports multilingual output, making diagnostic information accessible to a diverse healthcare workforce and patient population. Regular updates and validations of the models ensure that they remain accurate and effective as new medical data becomes available.

### Population Health Metrics and Medication Adherence Tracking Tool

In some example embodiments, this tool tracks key population health metrics and monitors medication adherence over time. The tool may include specific measures such as the Charm Score, which assesses morbidity and other health indicators. The system provides visualizations and reports that help healthcare providers understand the health status of individual patients and populations. This information may be used for planning interventions and improving overall health outcomes.

In some example embodiments, the medication adherence tracking component monitors patients'adherence to prescribed treatment regimens. An example component may collect data from various sources, including pharmacy records, patient self-reports, and electronic health records. In some embodiments, the system may identify patterns of non-adherence, such as missed doses or inconsistent medication use, and alert healthcare providers to potential issues.

In some example embodiments, the tool's population health metrics include measures of chronic disease prevalence, risk factors, and healthcare utilization. These metrics provide a comprehensive overview of population health trends, enabling healthcare organizations to identify areas of need and allocate resources effectively. The system also supports stratification of patients based on risk levels, allowing for targeted interventions that address the specific needs of different groups.

In some example embodiments, the method begins with the comprehensive collection of data related to population health metrics, such as chronic disease prevalence, demographic risk factors, and patterns of healthcare utilization. The system aggregates this data from various sources, including public health records, EHRs, and patient surveys, to provide a detailed overview of community health trends.

In some example embodiments, the system also tracks individual patient medication adherence by analyzing pharmacy fill records, electronic prescriptions, and patient-reported data. The system may identify patterns of adherence or non-adherence, such as consistently missed doses or inconsistencies in medication intake. This tracking may be enhanced by integrating real-time data, near real-time data, or substantially real-time data from wearable devices and other remote monitoring systems, which provide additional context and granularity.

In some example embodiments, healthcare providers receive detailed reports and visualizations that summarize both population health metrics and individual medication adherence patterns. These reports highlight high-risk patients or communities, enabling targeted interventions to improve health outcomes. For instance, the system can identify areas with low medication adherence rates and suggest public health campaigns or targeted outreach programs.

In some example embodiments, the system generates alerts for healthcare providers, e.g., when issues are detected, such as critical adherence issues may be detected, such as a sudden drop in adherence that could indicate a problem like side effects or access issues. Providers can use this information to engage with patients directly, offering support, counseling, or adjustments to their medication regimens. Patients may also be given access to a secure portal where they can track their own medication adherence, receive reminders, and access educational resources.

In some example embodiments, the tool supports secure communication between healthcare providers and patients, ensuring that sensitive information may be transmitted securely. An example tool may also provide predictive analytics capabilities, estimating future healthcare needs based on current trends and adherence patterns. This allows healthcare systems to allocate resources more effectively, plan for future demands, and implement preventive care strategies. Data privacy and compliance with healthcare regulations may be maintained through strict security measures and regular audits.

### Dynamic Clinical Workflow Automation and Task Management System

In some example embodiments, this system automates various clinical workflows and manages tasks dynamically based on real-time data, near real-time data, or substantially real-time data. An example system may adjust workflows according to the changing needs of patients and healthcare providers, ensuring efficient use of resources. The system includes task automation features, such as auto-scribing of clinical notes and automated scheduling of follow-up appointments. This automation reduces administrative workload and enhances the efficiency of clinical operations.

In some example embodiments, the dynamic aspect of the system allows the system to adapt to changing circumstances, such as new patient information or shifts in clinical priorities. The system may re-prioritize tasks, allocate resources, and update schedules in real-time, near real-time, or substantially real-time. This flexibility ensures that healthcare providers can respond quickly to emerging issues and provide timely care.

In some example embodiments, the system also includes advanced analytics capabilities, enabling healthcare providers to monitor workflow performance and identify areas for improvement. The system may provide insights into task completion times, resource utilization, and other key metrics. This data-driven approach supports continuous optimization of clinical workflows, improving efficiency and patient outcomes.

In some example embodiments, this method involves the automation of various clinical workflows and the dynamic management of tasks based on real-time data, near real-time data, or substantially real-time data. The system utilizes AI-driven processes to automate the generation of clinical documentation, such as progress notes, discharge summaries, and referral letters. This automation may be achieved by transcribing patient-provider interactions and structuring the information into standard medical formats.

In some example embodiments, the system schedules follow-up appointments and diagnostic tests automatically, based on clinical protocols and patient-specific data. For example, after a surgical procedure, the system will schedule necessary post-operative check-ups and imaging studies. This scheduling may be dynamic, allowing adjustments as new data may be received or patient conditions change. The system prioritizes tasks and allocates them to healthcare providers according to their expertise and availability, optimizing the use of resources.

In some example embodiments, a central task management interface provides healthcare providers with an overview of all ongoing tasks, including their status, priority, and due dates. The system sends alerts and reminders for tasks, e.g., critical tasks, ensuring that important actions are not overlooked. The central task manager may also allow providers to reassign tasks or adjust priorities as needed, providing flexibility to accommodate changes in patient needs or clinical workflows.

In some example embodiments, the system's analytics capabilities allow healthcare administrators to monitor workflow efficiency, track task completion rates, and identify bottlenecks. These insights can inform decisions about resource allocation, process improvements, and staff training needs. The system also supports secure communication between team members, facilitating the coordination of complex care plans and ensuring that all relevant information may be shared promptly.

In some example embodiments, compliance with healthcare regulations and data privacy standards may be a fundamental aspect of the system. All automated processes and task management activities may be logged, creating an audit trail that can be reviewed for compliance and quality assurance purposes. The system's design includes robust security measures to protect patient data, including encryption and access controls.

### Predictive Analytics System for Chronic Disease Management and Hospitalization Risk

In some example embodiments, the predictive analytics system uses advanced ML models to assess the risk of chronic diseases and potential hospitalizations. The system may analyze patient data, including medical history, lifestyle factors, and genetic information, to predict future health events. This system allows healthcare providers to identify high-risk patients early and implement preventive measures. The system's insights help in managing chronic conditions more effectively and reducing the likelihood of hospitalizations.

In some example embodiments, the system includes models for predicting the onset and progression of chronic diseases, such as diabetes, hypertension, and cardiovascular diseases. The system may consider various risk factors, such as age, family history, and lifestyle choices, to estimate the likelihood of disease development. The system also provides personalized recommendations for preventive care, such as lifestyle changes, regular screenings, and medication management.

In some example embodiments, in addition to disease management, the system assesses hospitalization risk by analyzing factors such as recent hospitalizations, comorbidities, and medication adherence. The system may predict the likelihood of readmission and suggest interventions to prevent it. The system's predictive capabilities enable healthcare providers to allocate resources more effectively, prioritize patient care, and improve overall health outcomes.

In some example embodiments, the method involves using predictive analytics to manage chronic diseases and assess the risk of hospitalization. The system collects comprehensive patient data, including clinical history, genetic information, lifestyle factors, and real-time, near real-time, or substantially real-time health metrics from wearable devices. This data may be processed using sophisticated machine learning models that predict the onset and progression of chronic diseases.

In some example embodiments, the predictive models consider a wide range of factors, such as demographic variables, known risk factors, and historical health data, to estimate the likelihood of disease development. For example, the system can predict the risk of diabetes onset in patients with prediabetes, allowing for early interventions. Similarly, the model may be used to assess the risk of complications in patients with chronic conditions like hypertension or chronic obstructive pulmonary disease (COPD).

In some example embodiments, healthcare providers receive these predictive insights through detailed reports that include specific recommendations for preventive care. These recommendations might include lifestyle modifications, preventive screenings, or adjustments to medication regimens. The system also generates alerts for patients at high risk of hospitalization, prompting proactive measures to prevent avoidable admissions.

In some example embodiments, the system's real-time analytics capabilities (or near real-time analytics capabilities, or substantially real-time analytics capabilities) allow for continuous monitoring of patient health, adjusting risk assessments as new data becomes available. For instance, changes in a patient's biometric data or new laboratory results can update the risk profile, triggering new recommendations or interventions. The system supports integration with EHR systems, ensuring that predictive analytics may be incorporated into the broader clinical workflow.

In some example embodiments, patients may be provided with personalized health education materials and preventive care resources through a secure portal. This portal also allows patients to track their health metrics, receive alerts about potential risks, and communicate with their healthcare providers. The system's data privacy and security measures ensure that all patient data may be protected, with strict adherence to healthcare regulations.

### Natural Language Processing Framework for Automated Clinical Documentation

In some example embodiments, the NLP framework automates the generation and summarization of clinical documentation. The system may be used to transcribe patient-provider interactions and generates comprehensive SOAP (Subjective, Objective, Assessment, Plan) notes that meet MEAT (Monitor, Evaluate, Assess, Treat) criteria. The system also provides language translation capabilities, ensuring that documentation may be accessible to a diverse patient population. This automation improves the accuracy and efficiency of clinical documentation, freeing up time for healthcare providers to focus on patient care.

In some example embodiments, the system's NLP capabilities extend beyond simple transcription, allowing the system to understand and extract key information from clinical conversations. The system may identify symptoms, diagnoses, treatment plans, and other relevant details, automatically populating the appropriate sections of the clinical record. The system also supports real-time, near real-time, or substantially real-time documentation, enabling healthcare providers to capture information during patient encounters.

In some example embodiments, in addition to generating SOAP notes, the system can summarize lengthy clinical documents, such as medical histories or consultation reports. An example system may highlight key findings and recommendations, making it easier for healthcare providers to review and understand complex cases. The system's language translation features ensure that clinical documentation may be available in multiple languages, supporting equitable care for patients from diverse linguistic backgrounds.

In some example embodiments, the method involves the use of a sophisticated NLP framework to automate the creation of clinical documentation. The system transcribes patient-provider interactions into structured clinical notes, such as SOAP (Subjective, Objective, Assessment, Plan) notes. An example system may use advanced NLP algorithms to identify and extract key information from the conversation, including patient symptoms, clinical observations, diagnoses, and treatment plans.

In some example embodiments, the extracted information may be organized into standardized formats that may be compliant with medical documentation requirements, such as MEAT (Monitor, Evaluate, Assess, Treat) criteria. The system's NLP capabilities include the ability to understand medical jargon, context-specific meanings, and the nuances of patient-provider communication. This ensures that the generated documentation may be accurate and comprehensive.

In some example embodiments, the system supports real-time, near real-time, or substantially real-time documentation, allowing healthcare providers to capture and review information during patient encounters. This feature may be particularly useful in fast-paced clinical environments, where timely documentation may be necessary. The system also offers multilingual support, enabling the documentation of patient encounters in multiple languages, which may be used for providing care to diverse patient populations.

In some example embodiments, in addition to generating standard clinical notes, the system can summarize long and complex clinical documents, such as comprehensive medical histories or detailed consultation reports. These summaries highlight key findings and recommendations, making it easier for providers to quickly understand the most relevant aspects of a patient's medical history.

In some example embodiments, healthcare providers can review, edit, and finalize the generated documentation through a secure interface. The system includes tools for adding annotations, making corrections, and entering additional observations. An example system may also integrate with EHR systems, ensuring that all documentation may be seamlessly incorporated into the patient's medical record.

In some example embodiments, to ensure the accuracy and quality of the documentation, the system includes feedback mechanisms that allow providers to report errors or inconsistencies. This feedback may be used to refine the NLP algorithms and improve the system's performance over time. Data privacy and security may be rigorously maintained, with encryption and access controls in place to protect patient information.

### AI-Enhanced Patient Engagement and Pre-Visit Preparation Tool

In some example embodiments, this tool enhances patient engagement by providing personalized information and preparing patients for their visits. An example system may use AI to analyze patient data and generate tailored pre-visit summaries, including medical history, upcoming appointments, and preparation instructions. The system also offers interactive features, such as appointment reminders and patient education materials. By improving patient engagement and preparation, the tool enhances the overall patient experience and supports better clinical outcomes.

In some example embodiments, the pre-visit preparation component provides patients with detailed information about what to expect during their visit, including any necessary preparations, such as fasting or bringing specific documents. An example system may also includes a checklist of questions patients may want to ask their healthcare providers, helping them make the most of their visit. The system can send reminders via email, SMS, or mobile app notifications, ensuring that patients may be well-informed and prepared.

In some example embodiments, the tool's patient engagement features extend beyond pre-visit preparation, offering ongoing support and education. An example system may provide information on managing chronic conditions, medication adherence, and lifestyle changes tailored to the individual patient's needs. The system also includes a feedback mechanism, allowing patients to provide input on their care experience. This feedback helps healthcare providers improve service quality and patient satisfaction.

In some example embodiments, the method for enhancing patient engagement begins with the analysis of patient data to generate personalized pre-visit summaries. These summaries include an overview of the patient's medical history, details about upcoming appointments, and specific preparation instructions, such as fasting before a blood test. The system uses AI to tailor this information to each patient's unique needs, ensuring that they may be well-prepared for their healthcare visits.

In some example embodiments, patients receive these summaries through multiple communication channels, including email, SMS, and mobile app notifications. The system sends reminders about appointment dates and any necessary preparations, such as bringing specific documents or medication lists. Patients can also confirm their appointments and submit any pre-visit questions they may have, facilitating better communication and preparation.

In some example embodiments, the system provides interactive features that enhance patient engagement, such as educational materials tailored to the patient's condition and treatment plan. These materials may include videos, infographics, and articles that explain medical procedures, medications, and lifestyle recommendations. The educational content may be designed to be accessible and easy to understand, empowering patients to take an active role in their healthcare.

In some example embodiments, a secure patient portal allows patients to access their medical history, view upcoming appointments, and track their healthcare progress. The portal includes tools for setting health goals, monitoring symptoms, and recording medication adherence. Patients can also provide feedback on their visit experience, which may be valuable for healthcare providers in improving service quality and patient satisfaction.

In some example embodiments, the system integrates with EHR systems, ensuring that all pre-visit preparation information may be available to healthcare providers. This integration allows providers to review the patient's pre-visit activities and any questions or concerns raised by the patient. The system supports secure communication between patients and providers, enabling the exchange of sensitive information in a protected environment.

In some example embodiments, to ensure data privacy and security, the system employs robust encryption methods and access controls. An example system may adhere to healthcare regulations and standards, protecting patient data at all stages of the engagement process. The system also includes compliance features that ensure all communications and data handling may be conducted in accordance with legal and ethical guidelines.

### Comprehensive Clinical Insights and Reporting System Utilizing AI and Machine Learning

In some example embodiments, this system provides comprehensive insights and reporting capabilities by leveraging AI and ML technologies. An example system may synthesize data from various sources, including EHRs, lab results, and patient-reported outcomes, to generate detailed reports. These reports highlight key clinical insights, trends, and patterns that inform decision-making. The system's analytics capabilities enable healthcare providers to identify areas for improvement and optimize clinical processes. This comprehensive reporting system supports continuous quality improvement and strategic planning in healthcare settings.

In some example embodiments, the system includes a dashboard interface that displays real-time data, near real-time data, or substantially real-time data and visualizations, such as charts and graphs, summarizing patient and population health metrics. Healthcare providers can customize the dashboard to focus on specific metrics, such as patient outcomes, treatment adherence, or resource utilization. The system also supports drill-down capabilities, allowing providers to explore data at different levels of detail.

In some example embodiments, in addition to standard reporting, the system offers predictive analytics and scenario modeling. An example system may simulate the impact of different treatment strategies, resource allocations, or policy changes, helping healthcare organizations make data-driven decisions. The system also includes benchmarking features, allowing organizations to compare their performance against industry standards or peer institutions. This comprehensive reporting and analytics capability supports informed decision-making and continuous improvement in healthcare delivery.

In some example embodiments, the method for generating comprehensive clinical insights involves collecting and analyzing data from a variety of healthcare sources, including EHRs, lab results, imaging studies, and patient-reported outcomes. The system uses AI and machine learning algorithms to process this data, identifying key trends, patterns, and insights that may be relevant to clinical decision-making.

In some example embodiments, the system generates detailed reports that summarize these insights, presenting them in a clear and actionable format. These reports include visualizations such as graphs, charts, and heatmaps that help healthcare providers understand complex data relationships and trends. The insights cover a wide range of clinical areas, from patient-specific treatment recommendations to broader population health metrics.

In some example embodiments, healthcare providers access these reports through a customizable dashboard interface. The dashboard allows providers to focus on specific metrics, such as treatment outcomes, resource utilization, and patient demographics. Providers can drill down into the data to explore specific cases or aggregate trends, enabling a deep understanding of clinical and operational performance.

In some example embodiments, the system also offers predictive analytics and scenario modeling capabilities. For example, the system may simulate the impact of different treatment strategies on patient outcomes, or model the effects of policy changes on healthcare costs and resource allocation. These predictive tools may be valuable for planning and decision-making, helping healthcare organizations optimize their strategies and improve patient care.

In some example embodiments, to ensure that the insights and reports may be actionable, the system includes benchmarking features that compare the organization's performance against industry standards or peer institutions. This benchmarking helps identify areas for improvement and best practices that can be adopted. The system supports secure communication and collaboration among healthcare providers, allowing them to discuss findings and develop coordinated action plans.

In some example embodiments, the system integrates with existing healthcare infrastructure, including EHR systems, to ensure seamless data flow and accessibility. An example system may adhere to strict data privacy and security protocols, protecting patient information and ensuring compliance with healthcare regulations. The system's comprehensive analytics and reporting capabilities support continuous quality improvement and strategic planning, and thus providing a tool for modern healthcare management.

### Combinations and Interactions

In some example embodiments, the described systems and tools can be combined and interact with each other to provide a seamless and integrated healthcare experience. For example, the Clinical Data Explorer can feed into the AI-Driven Treatment Recommendation System, providing relevant clinical data that informs treatment decisions. The Medication Adherence Monitoring System can work in tandem with the Predictive Analytics System to identify patients at risk of hospitalization due to non-adherence. The Ensemble ML Model Framework can support various subsystems by providing reliable predictions and analyses.

These integrated systems may enhance the overall functionality and effectiveness of the healthcare information technology platform, offering a holistic solution to the challenges faced by healthcare providers. The present disclosure provides a foundation for developing advanced, AI-driven tools that improve clinical decision-making, optimize workflows, and enhance patient care.

A processor-based system capable of implementing the described methods may include a sophisticated computing infrastructure designed to handle complex data processing, analysis, and communication tasks. This system integrates hardware and software components optimized for healthcare applications, ensuring efficiency, accuracy, and compliance with industry standards.

### Processor-Based System Overview

The processor-based system may be a highly configurable and scalable platform comprising multiple interconnected hardware and software modules. The system architecture may be designed to support the simultaneous processing of large datasets, real-time analytics, near real-time analytics, or substantially real-time analytics and secure data communication. An example system may be built on a robust hardware foundation that includes high-performance processors, memory units, storage devices, and network interfaces.

### Hardware Components

Central Processing Unit (CPU): The system may be equipped with a multi-core CPU, capable of executing complex algorithms and managing multiple tasks concurrently. The CPU may be designed for high-speed computation, which may provide for processing large volumes of healthcare data in real-time, near real-time, or substantially real-time.
Graphics Processing Unit (GPU): For tasks requiring intensive parallel processing, such as deep learning and image analysis, the system includes one or more GPUs. GPUs accelerate the processing of machine learning models, enhancing the system's ability to analyze medical images and large datasets efficiently.
Memory (RAM): The system features a large amount of RAM to support the rapid access and manipulation of data. This may provide for running AI and ML algorithms, which require substantial memory resources for model training and inference.
Storage Devices: The system includes high-capacity storage solutions, such as solid-state drives (SSDs) and hard disk drives (HDDs). These storage devices may be used to store patient data, medical records, and model parameters securely. The system employs redundant storage configurations (e.g., RAID) to ensure data integrity and availability.
Network Interface: To facilitate secure communication with other healthcare systems, the processor-based system includes high-speed network interfaces. These interfaces support wired (Ethernet) and wireless (Wi-Fi) connectivity, enabling the system to exchange data with EHR systems, medical devices, and cloud-based services.
Security Hardware: The system integrates specialized hardware components, such as trusted platform modules (TPMs) and hardware security modules (HSMs), to ensure secure data storage and encryption. These components protect sensitive patient information and ensure compliance with healthcare regulations.

### Software Components

Operating System (OS): The system runs on a secure and reliable operating system, such as a specialized healthcare-focused Linux distribution or a hardened version of Windows. The OS manages hardware resources, facilitates software execution, and ensures system security.
Data Integration Layer: A sophisticated data integration layer may be implemented to aggregate and standardize data from diverse healthcare sources, including EHRs, imaging systems, and patient-reported data. This layer includes data normalization, cleansing, and transformation capabilities, ensuring consistent data quality.
Artificial Intelligence and Machine Learning Frameworks: The system may be equipped with advanced AI and ML frameworks, such as TensorFlow, PyTorch, and scikit-learn. These frameworks provide the tools and libraries needed to develop, train, and deploy AI models for tasks such as predictive analytics, diagnostic suspecting, and treatment recommendation.
Natural Language Processing (NLP) Engine: An NLP engine may be integrated into the system to process and analyze unstructured text data, such as clinical notes and patient histories. This engine supports language models, text extraction, and summarization functionalities, enabling the system to generate structured clinical documentation and insights.
User Interface and Visualization Tools: The system includes a set of user interface components and visualization tools, enabling healthcare providers to interact with the system, access insights, and manage clinical workflows. These tools support customizable dashboards, real-time data visualization, near real-time data visualization, or substantially real-time visualization and interactive report generation.
Security and Compliance Software: To ensure data security and regulatory compliance, the system incorporates security software that provides encryption, access control, and audit logging. An example system may include features for secure data transmission, user authentication, and authorization, complying with standards such as HIPAA and GDPR.
Communication and Collaboration Tools: The system includes secure communication tools, such as encrypted messaging and video conferencing, to facilitate collaboration among healthcare providers. These tools may be integrated with the system's data management capabilities, allowing seamless sharing of patient information and clinical insights.
Data Analytics and Reporting Module: A comprehensive analytics and reporting module may be integrated into the system, providing advanced data analysis capabilities. This module supports the generation of detailed clinical reports, predictive models, and scenario simulations, aiding in clinical decision-making and strategic planning.

### System Capabilities

The processor-based system may be capable of implementing the described methods by leveraging such a system's hardware and software components. In some example embodiments, the system may support real-time data processing (or near real-time or substantially real-time) and analysis, enabling the generation of actionable clinical insights. The system can automate clinical workflows, manage complex tasks, and provide decision support through advanced AI and ML algorithms. An example system may facilitate secure data exchange with other healthcare systems, ensuring interoperability and seamless integration into existing clinical environments.

The system's architecture may be designed to be scalable and flexible in some example embodiments, allowing some example systems to adapt to the evolving needs of healthcare providers and patients. Some example embodiments may handle large volumes of data, accommodate new data sources, and integrate emerging technologies, such as telehealth and wearable devices.

### Security and Compliance

One aspect of the processor-based system may be the system's focus on security and compliance. The system employs state-of-the-art security measures to protect patient data, including encryption, secure access controls, and continuous monitoring. An example system may adhere to stringent healthcare regulations, ensuring that all data handling, storage, and communication practices meet legal and ethical standards. Regular security audits and updates may be conducted to address emerging threats and vulnerabilities.

This processor-based system represents a comprehensive solution for modern healthcare challenges, integrating advanced computing capabilities with specialized healthcare software. An example system may provide healthcare providers with the tools they need to deliver high-quality care, optimize clinical workflows, and make informed decisions. By leveraging cutting-edge technology, the system enhances patient outcomes, improves operational efficiency, and supports the secure and compliant management of healthcare data.

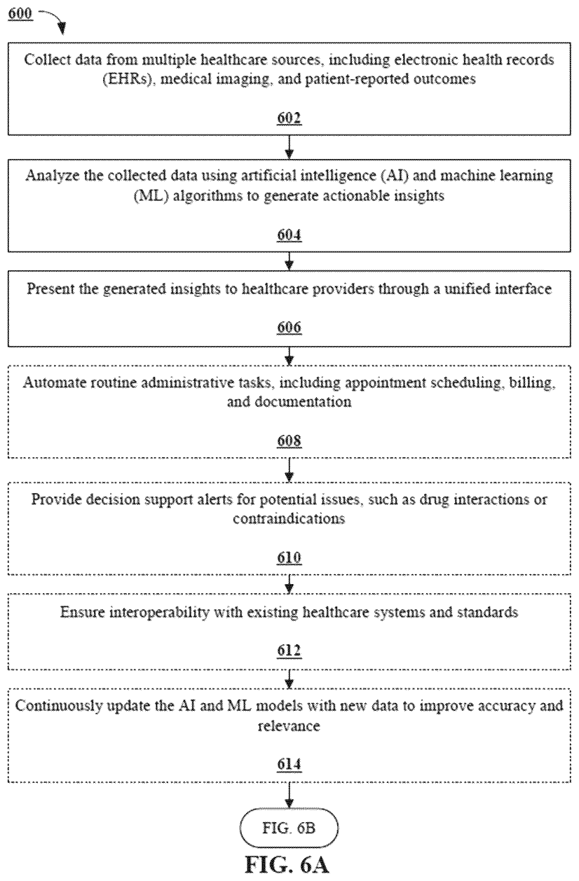

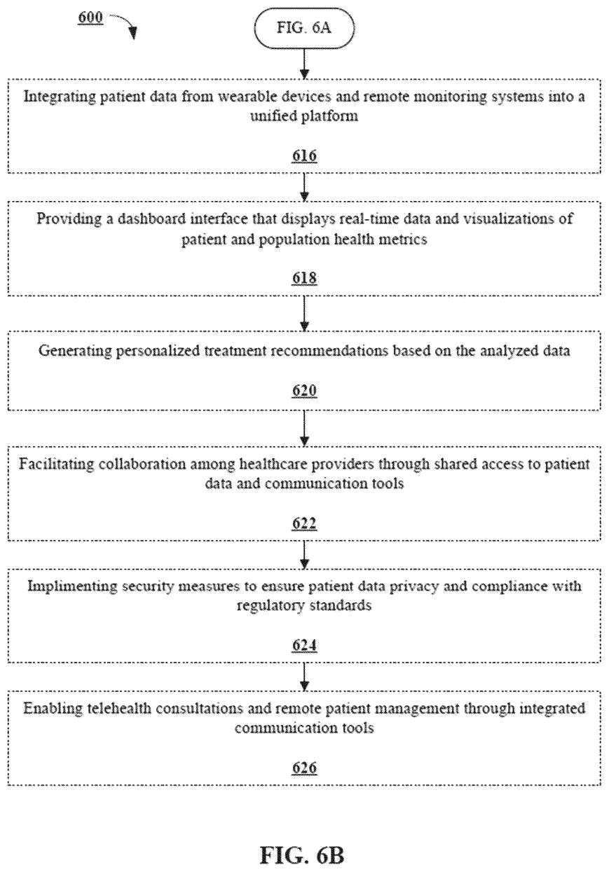

FIGS. 6A-6B illustrate a flow diagram for an example method of AI-driven collection, integration, and analysis of healthcare data from multiple sources to provide unified insights, automate tasks, and support clinical decision-making in accordance with the systems and methods described herein. Referring to FIG. 6A, the method involves collecting data from multiple healthcare sources, including electronic health records (EHRs), medical imaging, and patient-reported outcomes (602). After data collection, the method proceeds to analyzing the collected data using AI and ML algorithms to generate actionable insights (604). The generated insights may then be presented to healthcare providers through a unified interface (606). Optional steps include automating routine administrative tasks, such as appointment scheduling, billing, and documentation (608). The method may also provide decision support alerts for potential issues like drug interactions or contraindications (610). Ensuring interoperability with existing healthcare systems and standards may be another optional enhancement (612), as may be continuously updating the AI and ML models with new data to improve accuracy and relevance (614).

Referring to FIG. 6B, the method further optional steps include integrating patient data from wearable devices and remote monitoring systems into a unified platform (616) and providing a dashboard interface that displays real-time (or near real-time or substantially real-time) data and visualizations of patient and population health metrics (618). The method may also generate personalized treatment recommendations based on the analyzed data (620) and facilitate collaboration among healthcare providers through shared access to patient data and communication tools (622).

Implementing security measures to ensure patient data privacy and compliance with regulatory standards may be an additional optional step (624), as may be enabling telehealth consultations and remote patient management through integrated communication tools (626).

Collecting data (602) may involve accessing various databases, extracting relevant patient information, and converting data into a standardized format for further analysis. Analyzing the collected data (604) could include preprocessing to remove noise, training models on historical data, and testing algorithms for accuracy. Presenting the generated insights (606) might involve creating visualizations, generating reports, and ensuring that the interface may be intuitive and user-friendly.

Automating routine administrative tasks (608) may involve integrating automated systems with existing workflows, setting up triggers for routine tasks, and aligning processes with healthcare standards. Providing decision support alerts (610) could include integrating alert systems with patient records, calibrating thresholds for alerts, and validating their accuracy.

Ensuring interoperability (612) might involve mapping data formats, implementing middleware solutions, and conducting interoperability testing. Continuously updating AI and ML models (614) could include setting up automated data pipelines, retraining models on fresh datasets, and validating updated models.

Integrating patient data from wearable devices (616) may involve setting up data ingestion processes, synchronizing data from multiple devices, and converting data into actionable insights. Providing a dashboard interface (618) could involve designing the layout, integrating data streams, and setting up real-time data processing pipelines (or near real-time or substantially real-time).

Generating personalized treatment recommendations (620) might include identifying key patient parameters, using decision trees or other algorithms, and presenting these recommendations to healthcare providers. Facilitating collaboration among healthcare providers (622) could involve setting up secure data sharing protocols, integrating communication tools, and managing user permissions.

Implementing security measures (624) may involve encrypting data at rest and in transit, conducting security audits, and ensuring compliance with regulations. Enabling telehealth consultations (626) might involve integrating video conferencing tools, setting up remote monitoring systems, and ensuring compliance with relevant healthcare regulations.

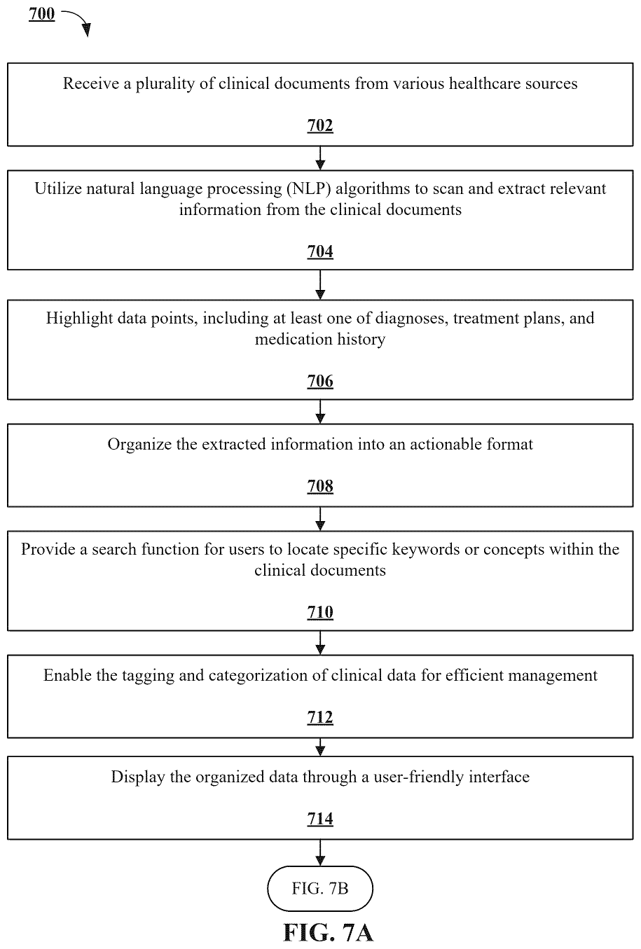

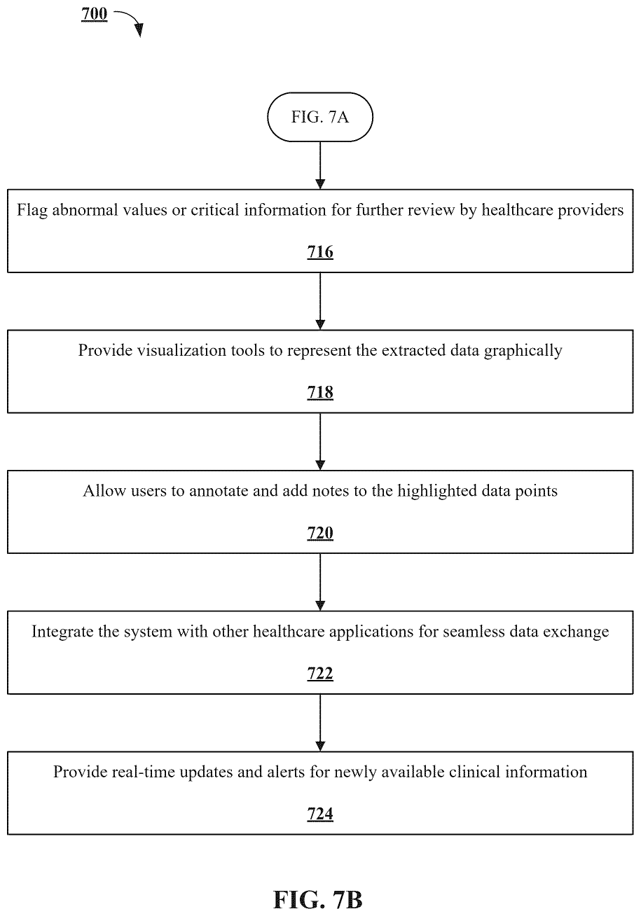

FIGS. 7A-7B illustrates a flow diagram 700 for an example method of NLP-powered extraction, organization, and presentation of key information from clinical documents with search, visualization, and alert features in accordance with the systems and methods described herein. More specifically, referring to FIG. 7A, flow diagram 700 illustrates a method for exploring and presenting clinical data, encompassing a series of steps designed to efficiently manage and display healthcare information. The process may begin with receiving a plurality of clinical documents from various healthcare sources (702) (although other orderings of steps are also possible). When the documents are collected, natural language processing (NLP) algorithms may be employed to scan and extract relevant information from these documents (704). The extracted information may be highlighted, focusing on data points such as one or more of diagnoses, treatment plans, and medication history (706). The highlighted data may be organized into an actionable format (708), which may make it easier for healthcare providers to utilize the information. The method may also include a search function that allows users to locate specific keywords or concepts within the clinical documents (710). Additionally, the data may be tagged and categorized for efficient management (712). The data may also be displayed through a user-friendly interface (714). One or more of these steps may be optional.

Referring to FIG. 7B, the method may also include other optional steps to enhance functionality. These optional steps involve flagging abnormal values or critical information for further review by healthcare providers (716), providing visualization tools to represent the extracted data graphically (718), enabling users to annotate and add notes to the highlighted data points (720), integrating the system with other healthcare applications for seamless data exchange (722), and offering real-time updates and alerts for newly available clinical information (724).

As discussed above, the method may start with receiving a plurality of clinical documents from various healthcare sources (702). This step may involve collecting clinical documents, including medical records, test results, and treatment histories, from multiple healthcare providers. Receiving the documents may also include processes such as authenticating the sources to ensure data integrity and transmitting the documents to the system.

Following this, natural language processing (NLP) algorithms may be used to scan and extract relevant information from clinical documents (704). The NLP algorithms may work by analyzing the text to identify information such as diagnoses, treatment plans, and medication history. This step may also include tokenizing the text into meaningful units and parsing it to recognize and classify the information based on context.

The method may highlight specific data points, including at least one of diagnoses, treatment plans, and medication history (706). Highlighting may involve visually marking or otherwise distinguishing these critical data points within the document to make them stand out. This may involve using flags, color coding, or other visual cues to emphasize important information for the user.

The extracted information may be organized into an actionable format (708). Organizing the information may involve summarizing patient histories, creating lists of treatment plans, and arranging the data in chronological order or by category. This may help ensure that the data is structured to be easily understood and effectively utilized by healthcare providers.

The method may also provide a search function for users to locate specific keywords or concepts within the clinical documents (710). This search function may allow users to quickly find relevant information by querying the database with specific terms related to their clinical interests. The search function may involve indexing the documents for faster retrieval and refining search algorithms to deliver the most relevant results.

Additionally, the method may enable the tagging and categorization of clinical data for efficient management (712). Tagging and categorization may involve assigning labels to various data points based on predefined rules or AI algorithms, which helps sort and organize the information for easy retrieval. This step may automatically classify data under relevant headings, such as ‘diagnoses,’ ‘treatment plans,’ or ‘medications.’

The organized data may be displayed through a user-friendly interface (714). This interface may allow users to interact with the data seamlessly, providing features like dashboards, data summaries, and customizable views. Depending on their needs, the display might support different layouts for different users, such as healthcare providers, administrators, or patients.

As an optional step, the method may include flagging abnormal values or critical information for further review by healthcare providers (716). This step involves identifying data points outside normal ranges or indicating potential health risks, which are then marked for immediate attention. The system might prioritize these flags based on the severity of the abnormality.

The method may also optionally provide visualization tools to represent the extracted data graphically (718). These tools may include charts, graphs, and other visual representations that make the data more accessible and interpretable. Visualization might involve plotting trends over time or showing correlations between different data points.

Another optional feature may include enabling users to annotate and add notes to the highlighted data points (720). This may allow healthcare providers to add their observations, comments, or recommendations directly onto the data points, facilitating better communication and decision-making.

The system may further integrate with other healthcare applications for seamless data exchange (722). This integration may help ensure that data can flow smoothly between systems, such as electronic health records (EHR), lab information systems (LIS), and pharmacy management systems. Integration may involve setting up APIs or other interfaces to enable real-time data sharing.

The method may also provide real-time updates and alerts for newly available clinical information (724). This may ensure that healthcare providers to have access to the most current data, which may help with timely decision-making. Implementing this step may involve setting up a monitoring system that constantly checks for updates and notifies users when new information becomes available.

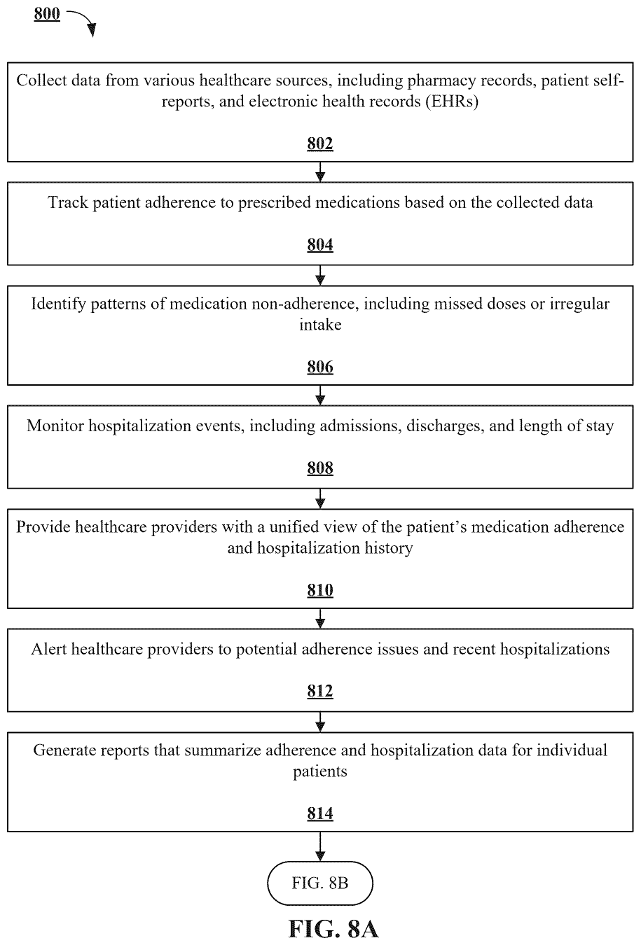

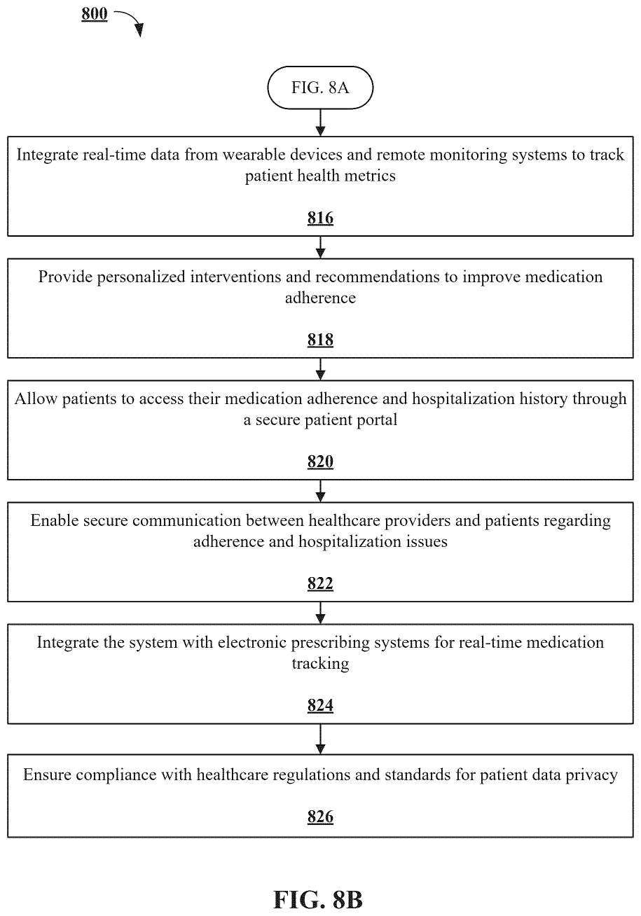

FIGS. 8A-8B illustrates a flow diagram 800 for an example method of monitoring medication adherence and hospitalization events to generate reports, provide alerts, and support interventions in accordance with the systems and methods described herein. More specifically, referring FIG. 8A, the method may begin by collecting data from various healthcare sources, including pharmacy records, patient self-reports, and electronic health records (EHRs) (802)(although other orders of the steps are possible). Additionally, the method may track patient adherence to prescribed medications based on this collected information (804). The method may identify patterns of medication non-adherence, such as missed doses or irregular intake (806). In addition to tracking adherence, the method monitors hospitalization events, including admissions, discharges, and length of stay (808). This combined data may provide healthcare providers with a unified view of the patient's medication adherence and hospitalization history (810). The method may also alert healthcare providers to potential adherence issues and recent hospitalizations (812). The method generates reports summarizing adherence and hospitalization data for individual patients (814).

In addition to these steps, the method includes several optional steps that enhance its functionality. For example, the method may integrate real-time data from wearable devices and remote monitoring methods to track patient health metrics (816). Referring now to FIG. 8B the method may also provide personalized interventions and recommendations to improve medication adherence (818). Patients can access their medication adherence and hospitalization history through a secure patient portal (820) in some embodiments. The method may further enable secure communication between healthcare providers and patients regarding adherence and hospitalization issues (822). Another optional step involves integrating the method with electronic prescribing methods for real-time medication tracking (824). The method may help ensures compliance with healthcare regulations and standards for patient data privacy and security (826).

The method may include collecting data from various healthcare sources, including pharmacy records, patient self-reports, and electronic health records (EHRs) (802). This step may involve gathering information from different sources to provide a comprehensive overview of a patient's medication history and health status. Collecting data may include verifying the accuracy of the data, consolidating the data from multiple databases, and standardizing the formats to ensure consistency and reliability.

The method may track patient adherence to prescribed medications based on the collected data (804). This step involves monitoring whether patients take their medications as prescribed, including the correct dosages and timing. Tracking adherence may include comparing medication use against prescribed regimens and flagging discrepancies. This tracking helps healthcare providers identify potential patient medication regimen issues.

The method may identify patterns of medication non-adherence, including missed doses or irregular intake (806). Identifying these patterns may involve analyzing the tracked data to detect deviations from the prescribed medication schedule. Analyzing the tracked data to detect deviations from the prescribed medication schedule may include recognizing repeated instances of missed doses, late doses, or incorrect dosages. By identifying these patterns, the method can help healthcare providers understand the root causes of non-adherence, whether they stem from forgetfulness, side effects, or other factors.

The method may monitor hospitalization events, including admissions, discharges, and length of stay (808). Monitoring hospitalization events may include recording each time a patient is admitted to or discharged from a hospital and the duration of their stay. This step may help to contextualize the patient's medication adherence within their broader health history and provides insights into how their adherence may impact their hospital visits.

With this comprehensive data, the method may provide healthcare providers with a unified view of the patient's medication adherence and hospitalization history (810). This step may aggregate the tracked adherence data and hospitalization events into a cohesive overview. Providing this unified view may include presenting the information in an easily accessible format, such as a dashboard or summary report, enabling healthcare providers to grasp a patient's overall health trajectory quickly.

To further aid healthcare providers, the method may alert them to potential adherence issues and recent hospitalizations (812). These alerts may notify providers of non-adherence patterns or hospital admissions that require immediate attention. Alerting healthcare providers may include sending notifications through the method's interface or other communication channels, ensuring timely intervention.

The method may generate reports summarizing adherence and hospitalization data for individual patients (814). These reports may include detailed summaries of a patient's medication adherence patterns, including missed doses and irregular intake, alongside their hospitalization history. Generating these reports may involve creating visual representations of the data, such as graphs or charts, to make the information more accessible and actionable for healthcare providers.

Beyond these steps, the method may integrate real-time data from wearable devices and remote monitoring methods to track patient health metrics (816). This integration may allow for continuous patient health monitoring, providing real-time updates that can be correlated with medication adherence and hospitalization events. Incorporating wearable data may enhance the method's ability to detect early signs of health deterioration or non-adherence.

The method may also provide personalized interventions and recommendations to improve medication adherence (818). These interventions could include tailored advice on managing side effects, reminders to take medications, or adjustments to the medication regimen based on the patient's unique circumstances. Providing these recommendations may involve analyzing the collected data to determine the most effective strategies for each patient.

Patients may be allowed to access their medication adherence and hospitalization history through a secure patient portal (820). This step enables patients to view their health data, empowering them to actively manage their health. Access through a secure portal may include features such as viewing past adherence patterns, understanding hospitalization events, and receiving reminders for upcoming doses.

The method further enables secure communication between healthcare providers and patients regarding adherence and hospitalization issues (822). This secure communication may facilitate discussions about any concerns the patient or provider might have, such as potential side effects, difficulty following the medication regimen, or follow-up care after a hospital discharge. Secure communication channels may include encrypted messaging methods within the patient portal or direct notifications from the healthcare provider.

Another optional step involves integrating the method with electronic prescribing methods for real-time medication tracking (824). This integration may allow for immediate updates to the patient's adherence records whenever a new prescription is issued, or an existing one is modified. Helping to ensure that the medication tracking method is up to date can help prevent gaps in adherence that may occur due to changes in the prescribed regimen.

Finally, the method ensures compliance with healthcare regulations and standards for patient data privacy and security (826). Compliance may involve implementing stringent data protection measures, such as encryption and access controls, to safeguard patient information. This step also includes regular audits and updates to the method to ensure it meets evolving regulatory requirements, thereby maintaining trust and security in its operation.

Flowchart 800 thus provides a comprehensive method for monitoring medication adherence and hospitalization events, with core steps such as collecting data (802), tracking adherence (804), and generating reports (814). Optional steps like integrating wearable data (816) and ensuring regulatory compliance (826) further enhance the method's functionality, making it a robust tool for improving patient care and outcomes.

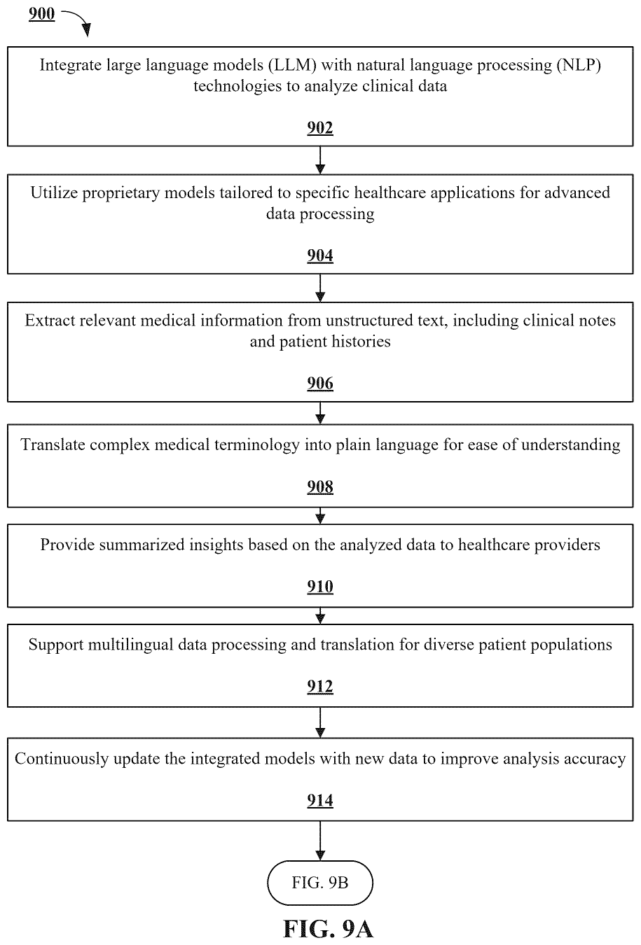

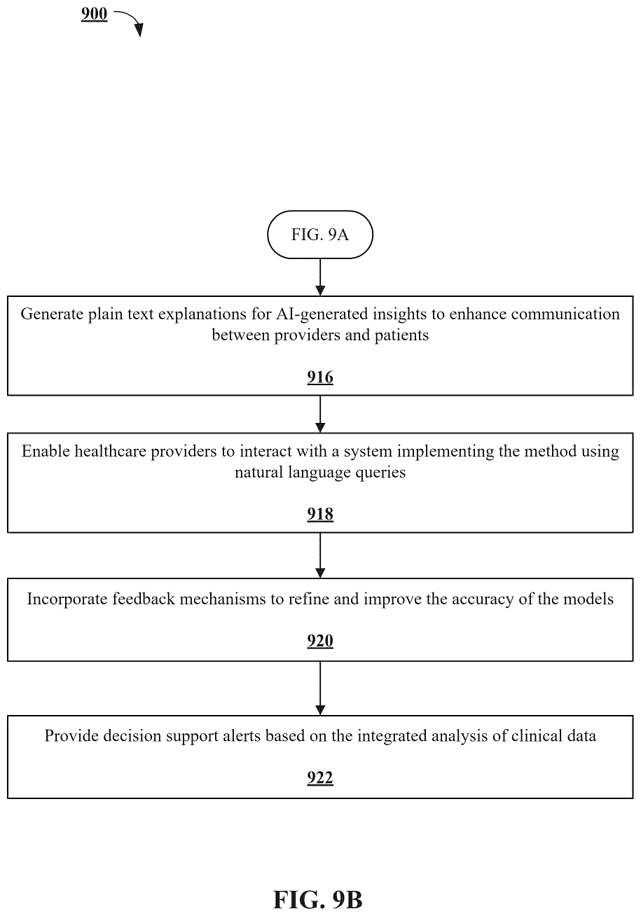

FIGS. 9A-9B illustrates a flow diagram 900 for an example method of using large language models with NLP to extract, translate, and summarize clinical information for multilingual, accessible decision support in accordance with the systems and methods described herein. More specifically, referring to FIG. 9A, the method may involve integrating large language models (LLM) with natural language processing (NLP) technologies to analyze clinical data (902). The method may utilize proprietary models tailored to specific healthcare applications for advanced data processing (904). The method may extract relevant medical information from unstructured text, including clinical notes and patient histories (906). This extracted information may be translated into plain language to ensure it is easily understood by healthcare providers and patients (908). The method may provide summarized insights based on the analyzed data to healthcare providers, making the information actionable (910). The method may support multilingual data processing and translation to cater to diverse patient populations (912). To maintain accuracy, the integrated models may be updated with new data (914). For example, the integrated models may be continuously updated with new data.

Referring to FIG. 9B, in addition to the above steps, the method includes several optional steps that may further enhance its functionality. For instance, the system may generate plain text explanations for AI-generated insights to enhance communication between providers and patients (916). Healthcare providers may interact with the system using natural language queries, allowing for intuitive data exploration and decision-making (918). The method may incorporate feedback mechanisms to refine and improve the accuracy of the models over time (920). Furthermore, decision support alerts may be provided based on the integrated analysis of clinical data, assisting healthcare providers in making timely and informed decisions (922). Systems implementing the method may include compliance measures for data privacy and security regulations to ensure that all data handling is conducted securely and within legal standards.

The method may begin by integrating large language models (LLM) with natural language processing (NLP) technologies to analyze clinical data (902) (although other orders of steps are also possible). This integration may involve combining the advanced language capabilities of LLMs with the specific domain expertise embedded in NLP technologies, allowing the system to effectively process and understand complex medical information. Integrating these technologies may include setting up a framework where the LLMs can interpret the nuances of medical language, while NLP technologies ensure that the context of healthcare-specific terminology is maintained throughout the analysis.

The method may utilize proprietary models tailored to specific healthcare applications for advanced data processing (904). These proprietary models may include specialized algorithms designed to address particular challenges in healthcare, such as diagnosing diseases or planning treatments. Utilizing these models may involve applying them to the clinical data to extract deeper insights, recognize patterns, and provide more accurate predictions. The proprietary nature of these models allows them to be highly optimized for specific use cases within the healthcare domain.

The method may then extract relevant medical information from unstructured text, including clinical notes and patient histories (906). This step may involve processing the raw text data to identify and isolate key information pertinent to patient care. Extracting this information may include parsing through extensive clinical notes, identifying critical details such as diagnoses, treatments, and patient responses, and organizing these details into a structured format that healthcare providers can easily access and utilize.

The method may translate complex medical terminology into plain language for ease of understanding (908). This translation may involve converting technical medical terms and jargon into language that is accessible to both healthcare providers and patients. By simplifying the language, the system helps ensure that the critical information is available and understandable, which may be used for effective communication and decision-making in healthcare settings.

The method may provide summarized insights to healthcare providers based on the analyzed data (910). These insights may include concise summaries of patient histories, potential diagnoses, treatment recommendations, and other relevant data points derived from the analysis. Providing these summaries may involve generating reports or visual dashboards that present the most critical information in a way that supports quick and informed decision-making.

Additionally, the method supports multilingual data processing and translation for diverse patient populations (912). This capability may involve processing and translating clinical data into multiple languages, ensuring that the system can be used effectively in multicultural and multilingual healthcare environments. Supporting multilingual processing may include both the analysis of data in various languages and the translation of outputs, making the system versatile and accessible to a broad range of users.

The integrated models may be updated with new data to ensure that the analysis remains accurate and relevant (914). For example, the integrated models may be continuously updated with new data. This continuous updating may involve regularly incorporating new medical research, patient data, and other relevant information into the models. By doing so, the system can adapt to new trends, medical discoveries, and evolving healthcare practices, thereby maintaining the precision and relevance of its analyses.

Referring to FIG. 9B, in addition to the above steps, the method includes several optional steps that may further enhance its functionality. For instance, the system may generate plain text explanations for AI-generated insights to enhance communication between providers and patients (916). Healthcare providers may interact with the system using natural language queries, allowing for intuitive data exploration and decision-making (918). The method may incorporate feedback mechanisms to refine and improve the accuracy of the models over time (920). Furthermore, decision support alerts may be provided based on the integrated analysis of clinical data, assisting healthcare providers in making timely and informed decisions (922). Systems implementing the method may include compliance measures for data privacy and security regulations to ensure that all data handling is conducted securely and within legal standards.

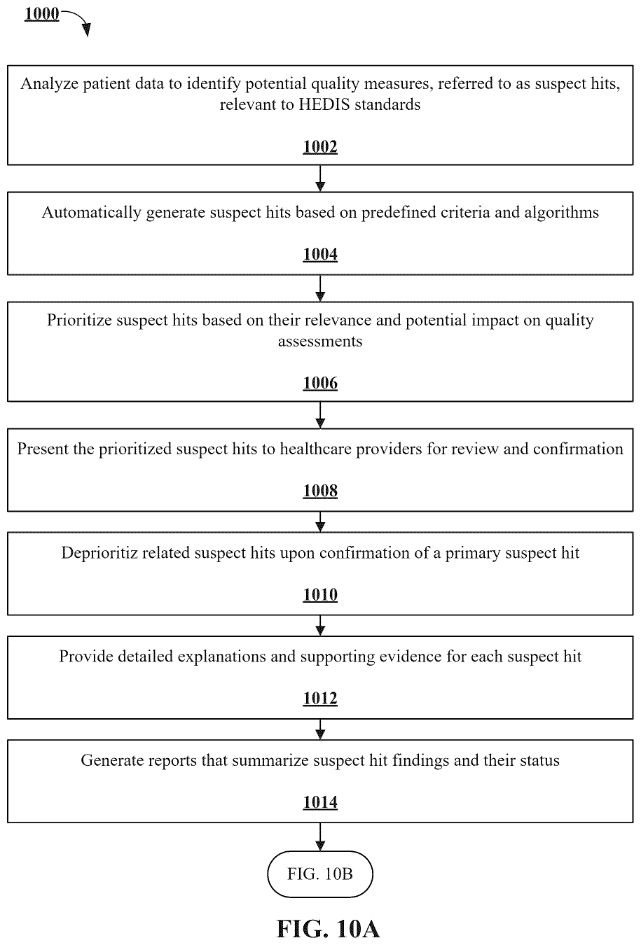

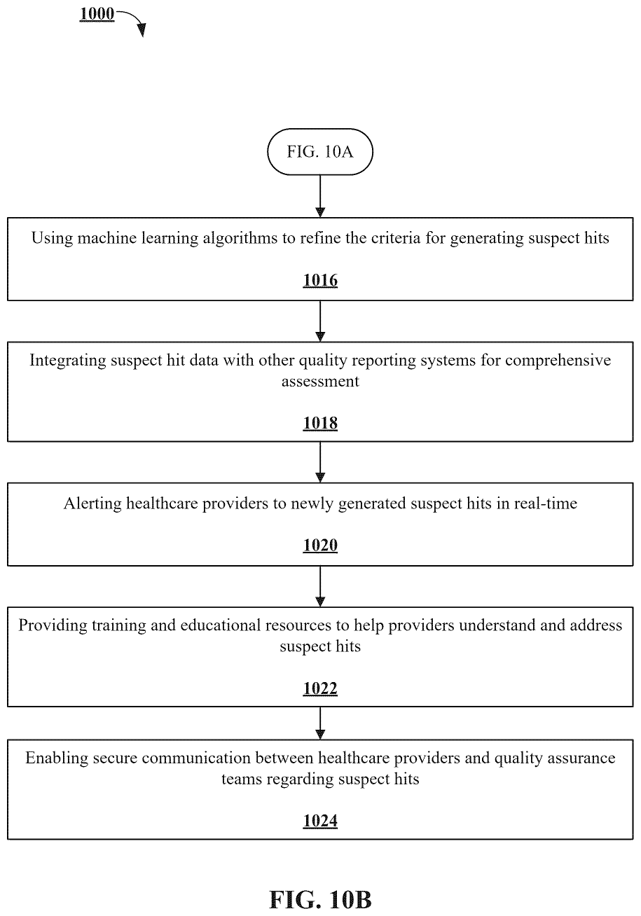

FIGS. 10A-10B illustrates a flow diagram 1000 for an example method of detecting, prioritizing, and reviewing quality-measure “suspect hits” for HEDIS compliance, with reporting and integration into quality workflows in accordance with the systems and methods described herein. More specifically, referring to FIG. 10A, the method may involve analyzing patient data to identify potential quality measures, referred to as suspect hits, that are relevant to HEDIS standards (1002). The method may automatically generate them based on predefined criteria and algorithms (1004). The method may also prioritize the suspect hits based on their relevance and potential impact on quality assessments (1006). These prioritized suspect hits may be presented to healthcare providers for review and confirmation (1008). Related suspect hits may be deprioritized (1010). The system may also provide detailed explanations and supporting evidence for each suspect hit to assist healthcare providers in their review (1012). The method may include generating reports that summarize the suspect hit findings and their status, providing a comprehensive overview of the quality assessment process (1014).

In addition to these steps, the method may include several optional steps to enhance functionality. For example, in FIG. 10B, the system may use machine learning algorithms to refine the criteria for generating suspect hits (1016). The method may also integrate suspect hit data with other quality reporting systems for a comprehensive assessment (1018). Further, healthcare providers may be alerted to newly generated suspect hits in real-time (1020). The method may include providing training and educational resources to help providers understand and address suspect hits (1022). Additionally, the method may include enabling secure communication between healthcare providers and quality assurance teams regarding suspect hits (1024).

The method may begin by analyzing patient data to identify potential quality measures, referred to as suspect hits, relevant to HEDIS standards (1002) (although other orders of steps are possible). This step may involve examining various patient data points, such as clinical records, treatment outcomes, and demographic information, to determine if they meet certain criteria that could indicate a potential quality issue. Analyzing the data may include running algorithms that cross-reference patient information with HEDIS standards, identifying any discrepancies or areas where care quality could be questioned.

The method may automatically generate suspect hits based on predefined criteria and algorithms (1004). This may involve applying specific rules and decision-making processes embedded within the system to flag these potential issues as suspect hits. Generating these hits may include evaluating the severity of the potential issue, its relevance to patient care, and its alignment with HEDIS standards. The automated nature of this step ensures that suspect hits are consistently identified across large datasets, reducing the risk of human error.

After suspect hits are generated, the method may prioritize them based on their relevance and potential impact on quality assessments (1006). Prioritizing suspect hits may involve assessing their significance relative to other findings, considering the potential risk to patient outcomes and the likelihood of recurrence. The method may rank the suspect hits, ensuring that healthcare providers bring the most critical issues to the forefront for immediate attention.

These prioritized suspect hits may be presented to healthcare providers for review and confirmation (1008). Presenting the suspect hits may include displaying them in a user-friendly interface, allowing healthcare providers to navigate the findings easily. Providers can review the suspect hits, assess their validity, and determine whether they should be confirmed as genuine quality issues or dismissed. This review process may be needed to ensure that only accurate and relevant hits are considered in the final quality assessment.

Upon confirmation of a primary suspect hit, the system may deprioritize related suspect hits (1010). This step may help to streamline the review process by reducing redundancy. Deprioritizing related hits may involve automatically adjusting their priority status in the system, ensuring that healthcare providers can focus on the most critical issues without being overwhelmed by duplicate or less significant findings.

To assist healthcare providers in their review, the system provides detailed explanations and supporting evidence for each suspect hit (1012). This supporting evidence may include references to relevant clinical guidelines, patient data, and other documentation that justifies the suspect hit. Providing these explanations ensures that healthcare providers have all the information they need to make informed decisions about each hit.

Finally, the method generates reports that summarize the suspect hit findings and their status (1014). These reports may include detailed summaries of the suspect hits, their prioritization, and the outcomes of the review process. Generating these reports may involve compiling the data into a format that is easy to interpret, such as charts, graphs, or tabular summaries. These reports provide a comprehensive overview of the quality assessment process, helping healthcare providers and administrators track performance and identify areas for improvement.

In addition to the above steps, the system may use machine learning algorithms to refine the criteria for generating suspect hits (1016). This optional step may involve continuously improving the algorithms that identify suspect hits, making them more accurate over time. Machine learning models may be trained on historical data to recognize patterns and adjust the criteria used to generate suspect hits, ensuring that the system adapts to new trends and changes in healthcare standards.

The system may also integrate suspect hit data with other quality reporting systems for a comprehensive assessment (1018). Integrating this data may allow healthcare providers to view suspect hits in the context of broader quality reporting efforts, providing a more holistic view of patient care. This integration may involve linking the suspect hits with other quality metrics, such as patient satisfaction scores or treatment outcomes, to create a complete picture of care quality.

Tools for healthcare providers to annotate and comment on suspect hits may be included in systems implementing the method. This feature allows providers to add their insights and notes directly to the suspect hits, facilitating communication and collaboration during the review process. Annotating the suspect hits may include adding context, raising questions, or suggesting further investigation, which can be valuable for ongoing quality improvement efforts.

The system implementing the method may include an audit trail for tracking the review and confirmation process. This audit trail ensures that all actions taken during the suspect hit review process are documented, providing transparency and accountability. Tracking the review process may involve recording who reviewed each suspect hit, what decisions were made, and when those decisions were finalized, creating a clear record for future reference.

In real-time, healthcare providers may be alerted to newly generated suspect hits (1020). This feature may help ensure that providers are immediately informed of any new suspect hits that require their attention. Alerting providers in real-time may include sending notifications through the system's interface, via email, or through other communication channels, ensuring that critical issues are addressed promptly.

To support healthcare providers, the system may provide training and educational resources to help them understand and address suspect hits (1022). These resources may include tutorials, guidelines, and best practices for handling suspect hits, ensuring that providers are well-equipped to manage the quality assessment process effectively.

The system implementing these methods may also include security features to protect patient data privacy. Protecting patient data may involve implementing encryption, access controls, and other security measures to ensure that sensitive information is securely stored and transmitted. This step may be needed to maintain compliance with healthcare regulations and ensuring that patient trust is upheld.

The method may enable secure communication between healthcare providers and quality assurance teams regarding suspect hits (1024). This communication feature allows for the secure exchange of information related to suspect hits, facilitating collaboration and ensuring that all relevant parties are informed and involved in the quality assessment process.

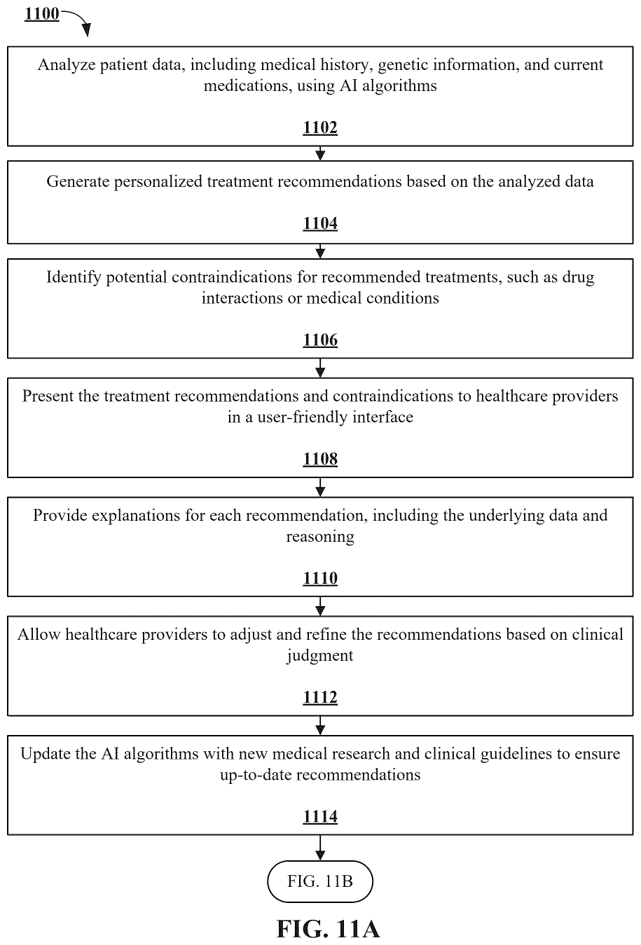

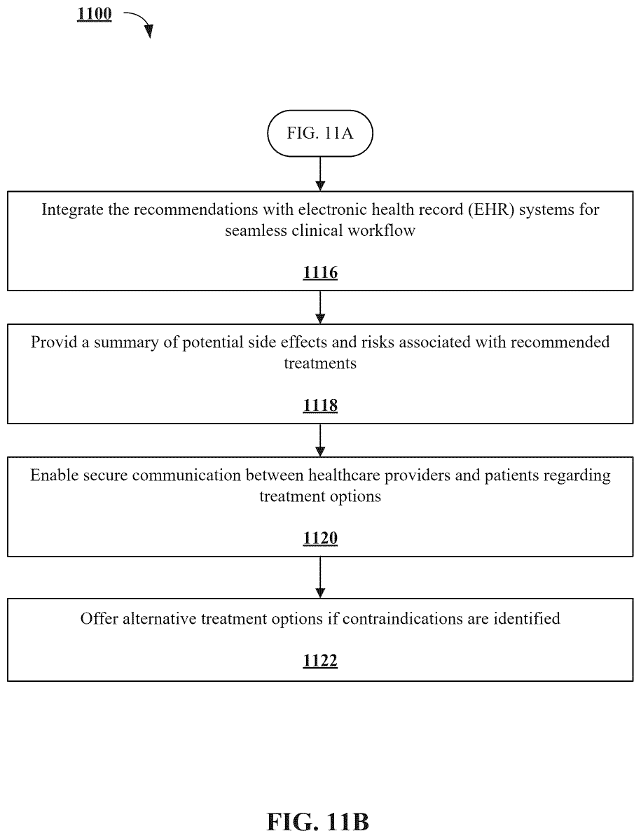

FIGS. 11A-11B illustrates a flow diagram 1100 for an example method of AI analysis of patient data to generate personalized treatment recommendations, identify contraindications, and provide actionable explanations in accordance with the systems and methods described herein. More specifically, referring to FIG. 11A, the method involves several steps. In one step, the method may analyze patient data, including medical history, genetic information, and current medications, using AI algorithms (1102). The method may generate personalized treatment recommendations based on the insights derived from the analysis (1104). The method may also identify potential contraindications for the recommended treatments, such as drug interactions or underlying medical conditions (1106). These treatment recommendations and identified contraindications may be presented to healthcare providers in a user-friendly interface (1108). Additionally, explanations may be provided for each recommendation, including the underlying data and reasoning behind them (1110).

In addition to these steps, the method includes several optional steps that enhance the functionality and usability of the process. For example, the method may include decision support alerts for critical contraindications, ensuring that healthcare providers are immediately informed of any significant risks (1112). Recommendations may be integrated with electronic health record (EHR) systems to ensure a seamless clinical workflow, allowing for easy access to and implementation of the suggested treatment plans (1116). The method may also support real-time updates and modifications to treatment plans, helping to ensure that the most current data is being used in decision-making (1114). Furthermore, the method may provide a summary of potential side effects and risks associated with the recommended treatments, allowing healthcare providers to make well-informed decisions (1118). In addition, explanations may include visual aids, such as charts and graphs, to illustrate key points, making the information more accessible and easier to understand (1110). Secure communication between healthcare providers and patients regarding treatment options may be enabled, ensuring that sensitive information is shared safely (1120). Multilingual support may be provided for both recommendations and explanations, making the method accessible to a diverse range of users (1120). If contraindications are identified, the method may offer alternative treatment options, ensuring that patients receive the safest and most effective care (1122). The method may also include compliance features for adherence to medical regulations and standards, ensuring that all recommendations and decisions are made within the bounds of legal and ethical guidelines (1122).

The method may begin by analyzing patient data, including medical history, genetic information, and current medications, using AI algorithms (1102). This analysis may involve collecting and processing a variety of patient data points to gain a comprehensive understanding of the patient's health profile. Analyzing the data may include identifying patterns, risks, and correlations that are not immediately apparent, providing a deeper insight into the patient's condition. This step may involve the use of advanced AI techniques to ensure that the analysis is both thorough and accurate.

The method may generate personalized treatment recommendations based on the analyzed data (1104). Generating these recommendations may involve selecting the most appropriate treatment options from a range of possibilities, tailored specifically to the patient's unique medical history and current condition. These recommendations may be informed by the latest clinical guidelines, research, and best practices in the medical field, ensuring that they are both relevant and effective.

The method may also identify potential contraindications for the recommended treatments, such as drug interactions or underlying medical conditions (1106). Identifying contraindications may involve cross-referencing the recommended treatments with the patient's existing medical conditions, medications, and genetic factors to ensure that no harmful interactions or adverse effects are likely to occur. This step may help safeguard patient health and ensure that the recommended treatments are effective and safe.

These treatment recommendations and identified contraindications may then be presented to healthcare providers in a user-friendly interface (1108). Presenting this information may involve organizing it in a clear, concise, and accessible manner, allowing healthcare providers to quickly grasp the most critical aspects of the recommendations and contraindications. The interface may be designed to facilitate easy navigation, ensuring that healthcare providers can efficiently review and act upon the information provided.

Additionally, explanations may be provided for each recommendation, including the underlying data and reasoning behind them (1110). Providing these explanations may involve detailing the rationale for each treatment recommendation, supported by the data and analysis that led to the conclusion. This step may help ensure transparency in the decision-making process, allowing healthcare providers to understand and trust the recommendations. The explanations may include references to relevant clinical guidelines, research studies, and patient-specific factors influencing the recommendations.

Integrating these recommendations with electronic health record (EHR) systems may facilitate seamless clinical workflows (1116). Integrating the AI-driven recommendations with EHR systems may streamline the clinical workflow by making the recommendations easily accessible within the provider's existing systems. This integration may involve automating data entry, allowing real-time updates, and ensuring compatibility with various EHR platforms. By embedding the recommendations directly into the clinical workflow, healthcare providers can implement the suggested treatments more efficiently, reducing the administrative burden and improving overall care delivery.

To support informed decision-making, the method may summarize potential side effects and risks associated with recommended treatments (1118). Summarizing the potential side effects and risks may allow healthcare providers to quickly assess each recommended treatment's benefits and drawbacks. These summaries may include information on common side effects, potential drug interactions, and specific risks based on the patient's medical history. By presenting this information in a concise and accessible format, the method helps providers make well-informed decisions that prioritize patient safety and efficacy.

Moreover, secure communication between healthcare providers and patients regarding treatment options is enabled, ensuring collaborative care (1120). The method may include secure channels for communication between healthcare providers and patients, allowing them to discuss treatment options in detail. This communication may involve encrypted messaging, secure video consultations, or other protected forms of interaction. By facilitating clear and confidential communication, the method may help ensure that patients are fully informed about their treatment plans and can actively participate in decision-making.

When contraindications are identified, the method may offer alternative treatment options to ensure patient safety and care continuity (1122). For example, when a contraindication is detected, the method may present alternative treatment options that are safer and more suitable for the patient. These alternatives may include different medications, non-pharmacological interventions, or adjustments to the treatment plan. Providing these options may help ensure that patient care remains flexible and adaptive, addressing any potential risks while maintaining effective treatment.

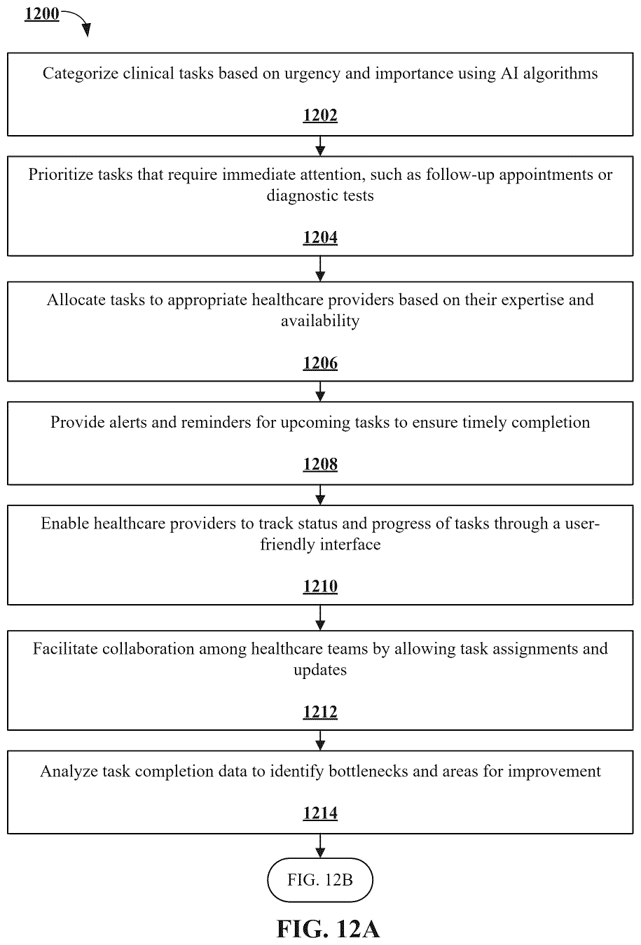

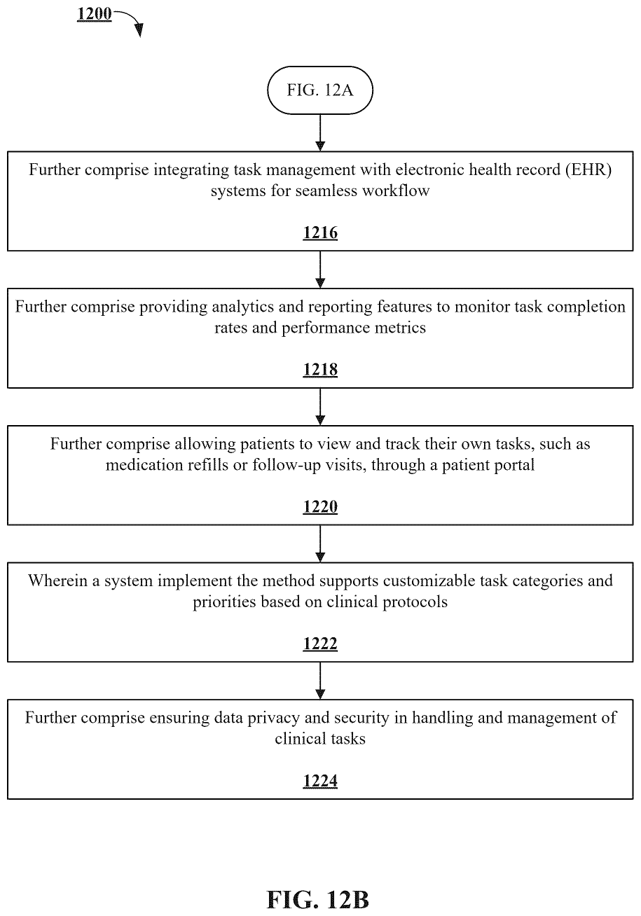

FIGS. 12A-12B illustrates a flow diagram 1200 for an example method of AI-driven clinical task management that categorizes, prioritizes, assigns, and tracks tasks, integrating with EHRs and supporting analytics in accordance with the systems and methods described herein. More specifically, referring to FIG. 12A, may include categorizing clinical tasks based on urgency and importance using AI algorithms (1202). It may also involve prioritizing tasks that require immediate attention, such as follow-up appointments or diagnostic tests (1204). Tasks may be allocated to appropriate healthcare providers based on their expertise and availability (1206), with alerts and reminders provided to potentially help ensure the timely completion of tasks (1208). Healthcare providers may track the status and progress of tasks through a user-friendly interface (1210). The method may further facilitate collaboration among healthcare teams by allowing task assignments and updates (1212) and may analyze task completion data to identify bottlenecks and areas for improvement (1214). Additionally, the method may include several optional steps to enhance the method's functionality, such as integrating task management with electronic health record (EHR) systems (1216). The method may include providing analytics and reporting features to monitor task completion rates and performance metrics (1218). The method may also allow patients to view and track their tasks, such as medication refills or follow-up visits, through a patient portal (1220). The method may include supporting customizable task categories (1222) and ensuring data privacy and security in handling and managing clinical tasks (1224).

The method may include categorizing clinical tasks based on urgency and importance using AI algorithms (1202). This step may involve analyzing factors such as patient condition, treatment protocols, and potential risks to determine each task's priority level. AI algorithms may evaluate the complexity and criticality of tasks, allowing for accurate and consistent categorization that reflects the current needs of the healthcare setting.

The method may also involve prioritizing tasks that require immediate attention, such as follow-up appointments or diagnostic tests (1204). This prioritization may involve selecting tasks that have the most significant impact on patient outcomes or time-sensitive ones. The process may include dynamically adjusting priorities as new information becomes available, ensuring that the most urgent tasks are addressed promptly.

Tasks may be allocated to appropriate healthcare providers based on their expertise and availability (1206). Allocating tasks may include matching tasks with providers with the relevant skills and experience and considering their current workload. This step may help ensure that tasks are performed efficiently and by the most qualified personnel, improving overall patient care.

The method may include providing alerts and reminders for upcoming tasks to ensure timely completion (1208). These alerts may be delivered through various channels, such as email, text messages, or in-app notifications, and may be customized based on the urgency and nature of the task. Reminders may help prevent tasks from being overlooked or delayed, contributing to better management of clinical workflows.

Healthcare providers may track the status and progress of tasks through a user-friendly interface (1210). The interface may allow providers to view ongoing tasks, monitor completion rates, and update the status of tasks in real-time. This tracking feature may include visual aids such as progress bars or color-coded indicators to quickly convey the current state of each task, facilitating effective task management.

The method may further facilitate collaboration among healthcare teams by allowing task assignments and updates (1212). Collaboration may involve enabling multiple providers to seamlessly assign tasks, share updates, and coordinate efforts. The system may include tools for real-time communication, document sharing, and status updates, ensuring that all team members are aligned and informed.

The method may analyze task completion data to identify bottlenecks and areas for improvement (1214). Analyzing this data may involve collecting and reviewing information on task completion times, frequency of delays, and resource utilization. The analysis may reveal patterns and trends that indicate inefficiencies or areas where processes could be streamlined, enabling continuous improvement in task management.

The method may include integrating task management with electronic health record (EHR) systems for seamless workflow (1216). Integration may involve synchronizing task data with patient records, allowing for automatic updates and reducing the need for manual data entry. This integration may ensure that task management is fully aligned with patient care plans and that all relevant information is accessible in one place.

The method may include providing analytics and reporting features to monitor task completion rates and performance metrics (1218). This step may involve collecting data on various aspects of task management, such as the time taken to complete tasks, the number of tasks completed within a specified timeframe, and the frequency of delays or missed deadlines. The analytics component may include generating visual reports, such as charts and graphs, highlighting trends and patterns in task performance, and enabling healthcare providers to identify areas where improvements may be needed quickly. Additionally, the reporting features may offer customizable dashboards that allow users to focus on specific metrics relevant to their roles, ensuring that the information provided is actionable and relevant to optimizing clinical workflows.

The method may also allow patients to view and track their tasks, such as medication refills or follow-up visits, through a patient portal (1220). This feature may involve providing patients with secure access to a user-friendly interface where they can monitor their ongoing healthcare tasks and receive reminders about upcoming actions they need to take. The patient portal may include tools for setting up alerts for tasks like medication refills, scheduling follow-up appointments, or completing health assessments, ensuring that patients remain engaged and proactive in managing their care. Additionally, the portal may offer options for patients to communicate with their healthcare providers, ask questions, or report any issues they encounter, further enhancing the collaboration between patients and their care teams.

The method may support customizable task categories and priorities based on clinical protocols (1222). Customization may allow healthcare providers to define task categories and priority levels that align with specific clinical guidelines or organizational needs. This flexibility may help ensure that the task management system is tailored to the unique requirements of the healthcare setting.

The method may also include ensuring data privacy and security in the handling and managing clinical tasks (1224). Ensuring privacy and security may involve implementing encryption, access controls, and audit trails to protect sensitive patient information. Compliance with relevant regulations and standards, such as HIPAA, may be a critical component of this step, safeguarding both patient data and the integrity of the task management process.

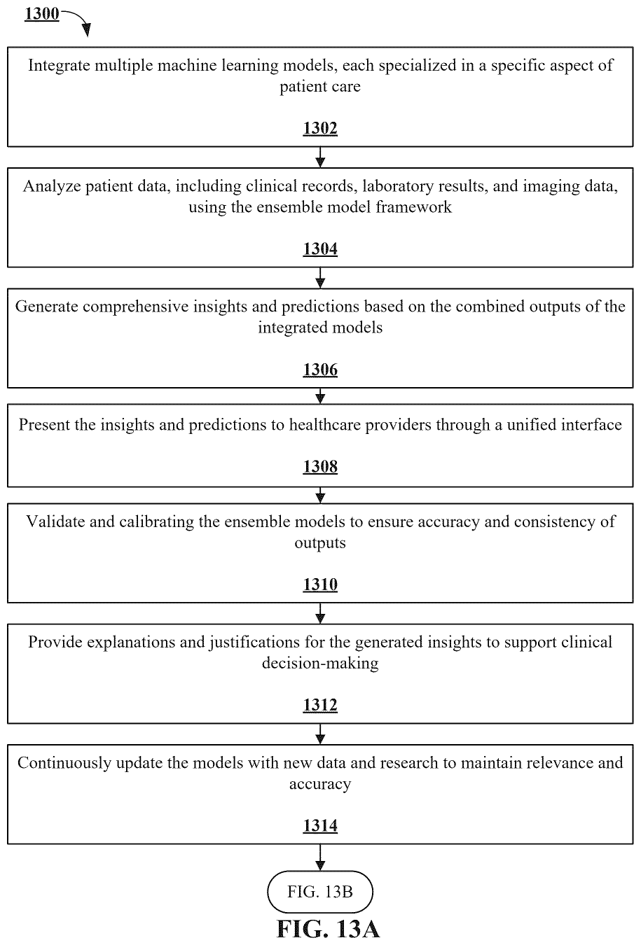

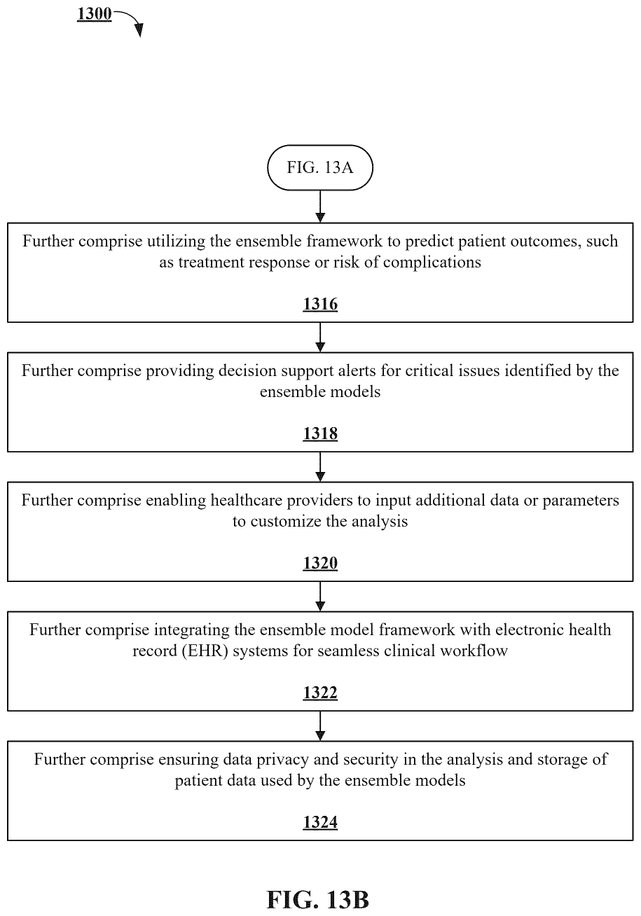

FIGS. 13A-13B illustrates a flow diagram 1300 for an example method to ensemble machine learning analysis combining multiple specialized models to generate predictions, insights, and decision support alerts in accordance with the systems and methods described herein. More specifically, referring to FIG. 13A, the method may involve integrating multiple machine learning models, each specialized in a specific aspect of patient care (1302). Using the ensemble model framework, the method may then analyze patient data, including clinical records, laboratory results, and imaging data (1304). In some examples, the method may generate comprehensive insights and predictions based on the combined outputs of the integrated models (1306). The method may include presenting insights and predictions to healthcare providers through a unified interface (1308).

Additionally, the method may involve validating and calibrating the ensemble models to ensure the accuracy and consistency of outputs (1310). To support clinical decision-making, the method may provide explanations and justifications for the generated insights (1312). The method may also continuously update the models with new data and research to maintain relevance and accuracy (1314). Optionally, the method may utilize the ensemble framework to predict patient outcomes, such as treatment response or risk of complications (1316). The method may also provide decision support alerts for critical issues identified by the ensemble models (1318). The method may integrate the ensemble model framework with electronic health record (EHR) systems to ensure seamless clinical workflow (1320). The method may ensure data privacy and security in the ensemble models'analysis and storage of patient data (1322).

The method may involve integrating multiple machine learning models, each specialized in a specific aspect of patient care (1302). This integration may involve selecting and combining models focused on distinct areas such as disease diagnosis, treatment planning, and risk assessment. The integration process may involve harmonizing the models'outputs to create a unified framework that may process and analyze a wide range of patient data. By drawing on the strengths of various models, the ensemble framework may provide a more comprehensive and accurate set of insights compared to any single model.

Using the ensemble model framework, the method may then analyze patient data, including clinical records, laboratory results, and imaging data (1304). Analyzing this data may involve several sub-processes, such as preprocessing the data to ensure compatibility with the models, feeding the data into the ensemble framework, and interpreting the combined outputs. The ensemble framework may leverage the unique capabilities of each integrated model to draw nuanced conclusions about the patient's condition, potential diagnoses, and treatment pathways. This step may ensure that all relevant aspects of patient data are thoroughly examined to provide a holistic view of the patient's health status.

Following the analysis, the method may generate comprehensive insights and predictions based on the combined outputs of the integrated models (1306). Generating these insights may involve synthesizing the outputs from each model to form coherent and actionable predictions about patient outcomes, such as treatment responses or risks of complications. This process may include cross-verifying results from different models to enhance reliability and applying statistical methods to quantify the certainty of the predictions. These insights may serve as a critical component in assisting healthcare providers with making informed clinical decisions.

The method may include presenting insights and predictions to healthcare providers through a unified interface (1308). Presenting this information may involve developing a user-friendly platform that organizes and displays the insights in a clear and accessible manner. This interface may include features allowing providers to navigate different insights quickly, filter results based on specific criteria, and view data visualizations that enhance their understanding of complex medical information. This step may help ensure that healthcare providers can efficiently access and utilize the insights generated by the ensemble model framework.

Additionally, the method may involve validating and calibrating the ensemble models to ensure the accuracy and consistency of outputs (1310). This validation process may include cross-validation techniques, where the models are tested against known outcomes to verify their performance. Calibration may involve adjusting the models'parameters to align their predictions with real-world data better. By continuously refining the models in this way, the method may help maintain high accuracy and reliability in the clinical insights provided.

To support clinical decision-making, the method may provide explanations and justifications for the generated insights (1312). Providing these explanations may involve detailing the rationale behind each prediction, supported by data from the patient's records and the analytical process used by the models. The explanations may be presented alongside the predictions in the user interface, ensuring that healthcare providers understand the reasoning behind the insights. This step may enhance transparency and trust in the system, allowing providers to make informed decisions based on the presented data.

The method may also continuously update the models with new data and research to maintain relevance and accuracy (1314). This update process may involve integrating the latest clinical research, treatment guidelines, and patient data into the ensemble framework. Regular updates may help ensure the models remain current and reflect the latest medical knowledge, improving their predictive power and relevance in clinical settings.

Optionally, the method may utilize the ensemble framework to predict patient outcomes, such as treatment response or risk of complications (1316). This predictive capability may be used for proactive patient care, allowing providers to anticipate potential issues and adjust treatment plans accordingly. The method may also provide decision support alerts for critical issues identified by the ensemble models (1318). These alerts may notify healthcare providers when the models detect significant risks, ensuring timely intervention and preventing adverse outcomes. Some example embodiments may enable healthcare providers to input additional data or parameters to customize the analysis (1320).

The method may integrate the ensemble model framework with electronic health record (EHR) systems to ensure seamless clinical workflow (1322). This integration may allow automatic data exchange between the ensemble framework and the EHR systems, enabling the models to access up-to-date patient information and update records with new insights. The method may ensure data privacy and security in the ensemble models'analysis and storage of patient data (1324). Ensuring privacy and security may involve implementing robust encryption, access controls, and compliance with healthcare regulations, safeguarding sensitive patient information.

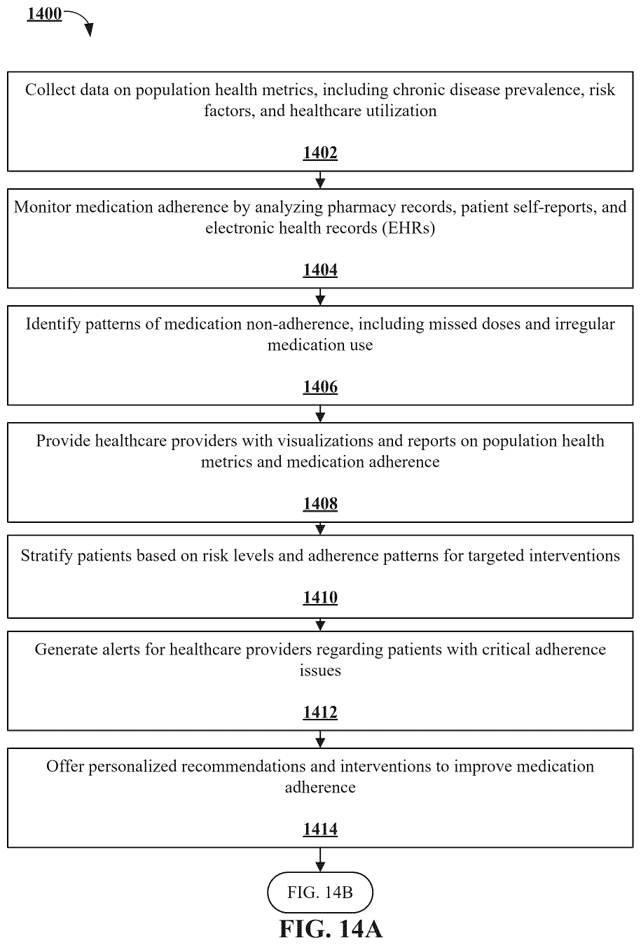

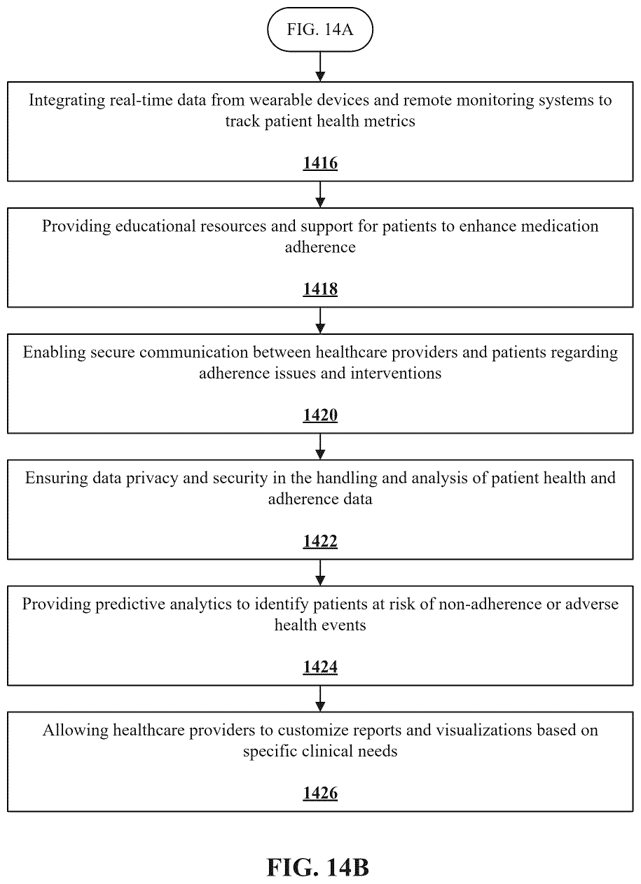

FIGS. 14A-14B illustrates a flow diagram 1400 for an example method of population-level health monitoring with adherence tracking, risk stratification, alerts, and personalized interventions in accordance with the systems and methods described herein. More specifically, referring to FIG. 14A, the method may involve collecting data on population health metrics, including chronic disease prevalence, risk factors, and healthcare utilization (1402). The method may also involve monitoring medication adherence by analyzing pharmacy records, patient self-reports, and EHRs (1404). In addition, the method may include identifying patterns of medication non-adherence, such as missed doses and irregular medication use (1406). The method may further provide healthcare providers with visualizations and reports on population health metrics and medication adherence (1408). Stratifying patients based on risk levels and adherence patterns for targeted interventions may also be a part of the method (1410). The method may include generating alerts for healthcare providers regarding patients with critical adherence issues (1412). Additionally, the method may involve offering personalized recommendations and interventions to improve medication adherence (1414).

Optional steps may include integrating real-time data from wearable devices and remote monitoring systems to track patient health metrics (1416). The method may also include tools for patients to track their own medication adherence through a secure portal (1418). Providing educational resources and support for patients to enhance medication adherence may be another optional step (1420). The method may ensure data privacy and security in the handling and analysis of patient health and adherence data (1422). The method may provide predictive analytics to identify patients at risk of non-adherence or adverse health events (1424). The method may allow healthcare providers to customize reports and visualizations based on specific clinical needs (1426).

The method may begin with collecting data on population health metrics, including chronic disease prevalence, risk factors, and healthcare utilization (1402). This data collection may involve gathering information from a variety of sources, such as public health records, patient surveys, and electronic health records (EHRs). The collected data may be used to create a comprehensive overview of the health status of a given population, providing insights into the prevalence of chronic diseases, the distribution of risk factors, and patterns of healthcare use.

The method may also involve monitoring medication adherence by analyzing pharmacy records, patient self-reports, and EHRs (1404). Monitoring adherence may include tracking whether patients are filling their prescriptions on time, taking their medications as directed, and reporting any difficulties they may have with their medication regimens. By comparing pharmacy records with patient self-reports and EHRs, healthcare providers may identify discrepancies and better understand adherence patterns.

In addition to monitoring adherence, the method may include identifying patterns of medication non-adherence, including missed doses and irregular medication use (1406). Identifying these patterns may involve analyzing data to detect instances where patients do not follow their prescribed medication regimens, whether due to forgetfulness, side effects, or other factors. Understanding these patterns may help healthcare providers identify patients who are at risk of poor health outcomes due to non-adherence.

The method may further provide healthcare providers with visualizations and reports on population health metrics and medication adherence (1408). These visualizations and reports may be designed to make complex data more accessible and actionable, allowing providers to grasp key trends and insights quickly. Visualizations might include graphs, charts, and heatmaps that show adherence rates across different population groups, track changes over time, or highlight areas of concern.

Stratifying patients based on risk levels and adherence patterns for targeted interventions may also be a part of the method (1410). Stratifying patients may involve grouping them according to their likelihood of experiencing adverse health outcomes based on factors such as their adherence patterns and underlying health conditions. This stratification may allow healthcare providers to prioritize their efforts and resources, focusing on patients most in need of intervention.

The method may include generating alerts for healthcare providers regarding patients with critical adherence issues (1412). These alerts may be triggered when the data indicates a patient is at immediate risk of negative health outcomes due to non-adherence. The alerts may be delivered through various channels, such as email, SMS, or within the healthcare provider's software interface, ensuring timely and appropriate responses.

Additionally, the method may involve offering personalized recommendations and interventions to improve medication adherence (1414). Offering these recommendations may include suggesting specific strategies for patients based on their individual adherence challenges, such as using pill organizers, setting reminders, or scheduling regular check-ins with healthcare providers. The goal may be to tailor interventions to each patient's unique circumstances to enhance adherence and improve health outcomes.

Optional steps may enhance the method's functionality. For example, the method may integrate real-time data from wearable devices and remote monitoring systems to track patient health metrics (1416). This integration may allow for continuous monitoring of patient's health, providing real-time insights into their condition and adherence to medication. The method may also include tools for patients to track their medication adherence through a secure portal (1418). These tools might offer features such as reminders, progress tracking, and access to educational resources, empowering patients to manage their health actively.

Providing educational resources and patient support to enhance medication adherence (1420) may be another optional step. Educational resources may include information on the importance of adherence, managing side effects, and tips for remembering to take medications. Support might involve access to healthcare providers for questions and concerns or participation in adherence support programs.

The method may also ensure data privacy and security in handling and analyzing patient health and adherence data (1422). Ensuring privacy and security may involve implementing encryption, secure access controls, and compliance with healthcare regulations such as HIPAA. This step may be used for maintaining patient trust and protecting sensitive health information.

Finally, the method may provide predictive analytics to identify patients at risk of non-adherence or adverse health events (1424). Predictive analytics may use machine learning algorithms to analyze historical data and predict which patients are most likely to experience adherence issues or suffer adverse health events. This predictive capability may allow healthcare providers to intervene proactively, addressing potential problems before they escalate.

The method may allow healthcare providers to customize reports and visualizations based on specific clinical needs (1426). This customization may involve selecting and tailoring the data presented in reports to focus on particular aspects of patient care, such as adherence trends, risk factors, or treatment outcomes. Providers may have the flexibility to adjust visual elements, like graphs and charts, to highlight information relevant to their clinical decisions. By enabling such customization, the method may help ensure that healthcare providers can access the most pertinent and actionable insights, enhancing the effectiveness of their patient care strategies.

In some examples, a system for enhancing clinical decision-making and workflow optimization may include one or more processors, system memory, and non-transitory computer-readable storage media storing instructions that, when executed, configure the processors to perform a set of coordinated functions. The system may incorporate data acquisition modules capable of securely connecting to multiple healthcare sources, such as hospital EHR servers, medical imaging archives, laboratory systems, and patient-facing mobile health applications. Data from these sources may include structured datasets, such as lab results and demographic profiles, as well as unstructured inputs, such as clinical notes and narrative discharge summaries.

A processing subsystem may execute artificial intelligence and machine learning models to analyze the collected data. These models may include supervised classifiers for risk scoring, natural language processing pipelines for unstructured text, and predictive analytics engines for outcome forecasting. An interface generation module may produce a unified display environment for healthcare providers, which may present patient summaries, alerts, and visual analytics in a clear and actionable format.

The system may also incorporate automation modules to handle administrative tasks, such as appointment scheduling, billing entry, and clinical documentation, including auto-scribing capabilities. Decision support subsystems may generate alerts for potential adverse drug interactions, contraindications, and other high-risk conditions, and predictive risk scoring engines may highlight patients likely to require urgent intervention.

Hardware interfaces and communication protocols may ensure interoperability with third-party healthcare IT systems via HL7, FHIR, and DICOM standards. The platform may include real-time model updating pipelines that automatically retrain AI/ML models using newly ingested patient data. The data ingestion subsystem may also capture biometric and activity data from wearable sensors and remote monitoring devices. A dashboard display generator may provide real-time visualization of both individual and population-level health metrics, and treatment recommendation modules may generate personalized care suggestions based on aggregated analysis. Collaboration tools may allow providers to share patient data and communicate securely, while embedded security mechanisms may enforce encryption, access control, and compliance with HIPAA and other regulatory frameworks. Integrated telehealth modules may enable secure video consultations and remote patient management.

A system for exploring and presenting clinical data may include a document ingestion subsystem configured to receive a plurality of clinical records from multiple healthcare data sources. These documents may include structured entries such as standardized lab reports and unstructured text such as physician notes and dictated summaries. A natural language processing engine may parse and extract relevant information, identifying items such as diagnoses, medication histories, and treatment plans.

A highlighting module may visually emphasize extracted data points in the original or reformatted text, while a data organization component structures the extracted information into actionable formats, such as chronological case histories or condition-specific overviews. A search and indexing engine may allow users to locate specific concepts or keywords within the corpus of documents.

Automated tagging and categorization subsystems may apply metadata to documents and data points according to predefined rules or AI classification algorithms. Visualization modules may render graphs, timelines, and other graphic representations of clinical data. An annotation interface may allow authorized users to append comments and notes to specific highlights, and multilingual support modules may translate data displays for accessibility in diverse settings. A real-time update engine may integrate new clinical information as it becomes available, issuing alerts to relevant users.

A system for monitoring medication adherence and hospitalization events may comprise a multi-source data acquisition unit configured to pull data from pharmacy dispensing records, patient self-reporting applications, and EHR prescribing logs. A medication adherence tracking engine may compare prescription instructions to actual refill and usage data, identifying patterns such as missed doses, delayed refills, or irregular consumption.

An event monitoring module may capture hospitalization events, including admissions, discharges, and length-of-stay records, from connected hospital information systems. A data fusion engine may combine adherence records and hospitalization histories into a unified patient profile, which may be presented to providers via a secure dashboard. A rule-based or AI-driven alerting system may notify clinicians of emerging adherence problems or recent hospitalizations.

The system may also include a report generation subsystem for creating visual summaries of adherence trends and hospitalization frequency. Integration with wearable sensors and remote monitoring devices may allow real-time tracking of health metrics, such as heart rate or blood glucose, correlated with medication use. Personalized intervention modules may generate targeted adherence improvement recommendations, while secure patient portals may allow individuals to view their own adherence histories. Secure communication channels may facilitate messaging between providers and patients, and e-prescribing integrations may provide real-time medication status updates. Security frameworks may enforce compliance with healthcare privacy and security regulations.

A system for enhanced healthcare data analysis may integrate large language models with natural language processing technologies in a combined computational environment. Proprietary domain-specific models may be deployed to address particular healthcare applications, such as oncology treatment planning or chronic disease management.

The system may include an unstructured text processing engine capable of extracting key medical information from clinical notes, correspondence, and patient histories. A terminology translation module may convert specialized medical language into plain-language explanations suitable for patients or non-specialist providers. Insight summarization modules may distill large volumes of data into concise, actionable outputs.

Multilingual processing engines may allow ingestion and analysis of data in multiple languages, with translation capabilities for output. Feedback capture mechanisms may store user edits and corrections for retraining analytical models. A decision support alerting component may generate clinical warnings based on integrated analysis, and a secure storage layer may archive analyzed datasets in compliance with privacy regulations. The system's interface layer may allow both structured form interaction and natural language queries from healthcare providers.

A system for automating suspect hit processing in quality reporting may include data analysis modules configured to compare patient records against HEDIS measure definitions. A rule-based or AI-driven generation engine may create suspect hit entries when measure criteria appear unmet.

A prioritization module may rank suspect hits according to potential quality impact, while a presentation interface may display ranked lists to provider reviewers. A dependency management engine may deprioritize related hits once a primary hit is confirmed. An evidence assembly module may link suspect hits to relevant clinical guidelines and supporting patient data. A reporting subsystem may summarize suspect hit status and resolution.

The system may incorporate machine learning refinements to adjust suspect hit generation criteria, annotation interfaces for provider comments, and integration points with other quality reporting tools. Real-time alerting mechanisms may notify staff of new high-priority hits. Security modules may protect patient data during processing, and secure communications interfaces may facilitate collaboration between providers and quality teams.

A system for generating AI-driven treatment recommendations may include a patient data aggregation engine capable of integrating medical history, genetic data, and current medication records. An AI recommendation engine may analyze this data against clinical guidelines and current research to produce personalized treatment suggestions.

A contraindication detection module may cross-reference recommended treatments with patient-specific risks, such as known allergies or potential drug interactions. A presentation interface may display recommendations alongside contraindication warnings, explanatory narratives, and visual aids like charts and graphs. Integration modules may connect recommendation output directly into EHR workflows.

Secure messaging systems may enable provider-patient discussions about treatment options, and multilingual output generators may produce recommendations and explanations in multiple languages. The system may also present alternative treatments when contraindications are detected. Compliance management features may ensure adherence to medical regulations and data privacy standards.

A system for task triage in disease management may incorporate AI-driven classification engines to categorize tasks by urgency and importance. A task prioritization module may queue high-impact actions, such as urgent follow-ups or abnormal lab reviews, ahead of routine activities.

Task allocation modules may assign work items to providers based on expertise, availability, and workload metrics. Alerting and reminder subsystems may deliver notifications through email, SMS, or in-app messages. A collaborative interface may allow real-time updates, task reassignment, and shared progress tracking.

Analytics engines may track completion rates, identify bottlenecks, and generate performance reports. Integration layers may link the task manager to EHR systems, ensuring synchronization with patient records. Patient-facing portals may allow individuals to view and manage their own assigned tasks, while customization controls may allow clinics to define task categories according to protocol. Security components may protect task-related patient data.

A system for ensemble-based clinical decision support may integrate multiple specialized machine learning models into a unified analysis framework. Data preprocessing modules may standardize inputs from clinical records, laboratory information systems, and imaging archives.

An ensemble aggregation engine may merge outputs from the specialized models to produce comprehensive insights, such as outcome predictions or treatment effect estimations. Model calibration modules may validate and adjust each model's performance to ensure consistent accuracy. An explanation generator may present rationale and supporting evidence for each insight, possibly with visual elements.

Optional predictive analysis modules may forecast patient outcomes, and decision support alert engines may notify providers when critical risks are detected. User input components may allow clinicians to refine model parameters or add supplemental patient data. Integration adaptors may connect the framework to EHR systems, while secure data storage and processing safeguards maintain regulatory compliance.

A system for population health and adherence monitoring may include large-scale data ingestion components capable of collecting health metrics such as disease prevalence, risk factor distribution, and healthcare utilization rates. Medication adherence monitoring modules may compare pharmacy transaction data, patient self-reports, and EHR prescription records.

An analytics subsystem may detect patterns of non-adherence and stratify patients into risk categories. Visualization tools may generate reports and dashboards for providers, showing adherence trends by population segment and over time. Alerting engines may flag patients with urgent adherence concerns.

Personalization modules may recommend targeted interventions for each patient, while optional integration with wearable devices and remote monitoring systems may enable real-time health tracking. Patient portal features may allow self-monitoring of adherence, access to educational resources, and secure communication with providers. Predictive analytics components may forecast which patients are at risk of poor outcomes, and reporting tools may be customizable to suit different clinical contexts. Security and privacy enforcement modules may ensure that all data handling meets applicable regulations.

In some embodiments, the systems described herein may be implemented as an integrated hardware and/or software platform configured to perform one or more of the functions of the methods of FIGS. 6A-6B to 14A-14B within a unified environment, or a subset of such functions. The platform may be deployed entirely on-premises within a healthcare facility, entirely in a cloud computing environment operated by a third-party infrastructure provider, or in a hybrid arrangement in which certain processing and storage functions are performed locally while others are executed remotely. In some examples, cloud-based clusters may be hosted in secure, compliant cloud infrastructures with geographically distributed servers to ensure availability and scalability, whereas on-premises implementations may utilize rack-mounted servers in a controlled data center environment. Hybrid deployments may also be employed, with patient-facing modules such as dashboards and telehealth services executed in the cloud while sensitive data processing modules remain on-premises for compliance and latency control. These hardware components collectively implement the functionality described in the methods of FIGS. 6A-6B to 14A-14B, such that the system performs the described data collection, analysis, and presentation steps within a physically deployed computing infrastructure, thereby linking the claimed methods to tangible, non-generic computing hardware.

The platform may include one or more processors configured to execute instructions for data collection, analysis, visualization, and communication across multiple functional modules. These processors may include general-purpose central processing units (CPUs) such as multi-core x86 or ARM processors to operate server operating systems, database management systems, and general application logic. In certain implementations, the processors may further include graphics processing units (GPUs) or tensor processing units (TPUs) to accelerate artificial intelligence (AI) and machine learning (ML) computations, including deep learning model training and inference for image analysis, natural language processing, and predictive analytics. Field-programmable gate arrays (FPGAs) may be incorporated to enable custom acceleration of specialized algorithms, such as signal processing for wearable device data or real-time encryption and decryption, while digital signal processors (DSPs) may be used for efficient handling of high-throughput data streams such as medical imaging files or continuous ECG signals. The processors may be housed in server-grade enclosures, blade server chassis, or compact embedded computing modules depending on deployment needs.

System memory may include volatile memory such as dynamic random-access memory (DRAM) for temporary storage of actively processed data, and non-volatile storage such as solid-state drives (SSDs), flash memory, or magnetic hard disk drives for persistent storage. Storage subsystems may be organized into database servers for structured patient records—using relational or non-relational database formats—and object storage repositories for large unstructured datasets such as medical imaging archives, scanned documents, or stored AI model files. Certain storage areas may be encrypted, with cryptographic keys stored within hardware security modules (HSMs) to maintain compliance with applicable privacy and security regulations.

Networking capabilities may include both wired and wireless interfaces. Wired connections may include Ethernet interfaces operating at gigabit or multi-gigabit speeds, as well as fiber optic connections for high-bandwidth local or wide-area communication. Wireless connections may include Wi-Fi 6, Bluetooth Low Energy (BLE), Zigbee, LTE, and 5G radios for integration with mobile devices, wearable devices, and remote monitoring equipment. Network topologies may be configured such that application servers, database servers, and interface servers are connected via a local area network (LAN) within a healthcare institution, with secure links to external networks for interoperability. Secure data transfers may be implemented using virtual private networks (VPNs), transport layer security (TLS) encryption, and mutual authentication protocols.

The platform may include one or more interface devices for interacting with healthcare providers and patients. Examples may include desktop workstations equipped with high-resolution displays for detailed medical imaging review, portable laptop computers, tablet devices for point-of-care decision-making, and wall-mounted touchscreen kiosks for patient check-in. Telehealth stations may be equipped with integrated cameras, microphones, biometric input devices, and dedicated lighting to facilitate remote consultations. Voice-enabled smart terminals may be deployed in sterile or high-activity environments to allow hands-free operation, while secure patient portals and mobile applications may allow patients to review health information, medication adherence history, and educational resources. Input devices may include keyboards, mice, medical-grade touchscreens, or voice recognition systems, while output devices may include visual displays, auditory alert systems, and haptic feedback mechanisms.

In some examples, the platform may integrate data from external sensors and wearable devices. Wearable devices may include smartwatches, fitness trackers, adhesive biosensors, or medical-grade continuous monitoring patches capable of measuring parameters such as heart rate, oxygen saturation, blood pressure, temperature, or glucose levels. Remote monitoring equipment may include home-based devices such as wireless weight scales, spirometers, or blood pressure cuffs. These devices may communicate with the platform using wireless protocols such as BLE, Zigbee, Wi-Fi, or cellular data connections. In certain cases, remote monitoring devices may transmit patient measurements at predefined intervals or in response to threshold events. Data may be buffered locally and batch-transmitted when network connectivity becomes available. The incoming data may be received by a secure gateway device that validates, encrypts, and forwards the data to analysis modules. The secure gateway may be implemented as a standalone appliance, a software client on a general-purpose computer, or an embedded module in a network router.

The hardware environment may incorporate security features at multiple levels. Hardware security modules may be used for encryption key management. Secure boot processes and firmware verification mechanisms may be implemented to prevent unauthorized system modifications. Biometric authentication devices, such as fingerprint readers or facial recognition cameras, may be used for user identity verification. Dedicated logging appliances may generate and store tamper-evident audit records to track system access and configuration changes.

In some embodiments, wearable or remote monitoring devices may incorporate self-calibration routines or baseline reference measurements to ensure sensor accuracy over time. For instance, a continuous glucose monitoring patch may periodically compare its readings against a capillary blood sample value, adjusting calibration coefficients to maintain accuracy within ±2% under normal operating conditions.

Specialized clinical data interfaces may be included to enable integration of data from multiple medical devices and sources. Medical device integration hubs may ingest data from ventilators, infusion pumps, and bedside monitors. Wearable device gateways may receive and normalize data from heart rate monitors, glucose sensors, smart scales, and activity trackers. Remote camera and imaging devices may capture data for telemedicine and remote diagnostics. Natural language processing accelerators may process large volumes of unstructured text from clinical notes, discharge summaries, and scanned documents. For example, a wearable ECG patch may capture a continuous 12-lead waveform and transmit the data via Bluetooth Low Energy to a secure gateway appliance located in the patient's home. The gateway may encrypt the data using AES-256, transmit it over a hospital's VPN connection to a GPU-equipped processing server, where an AI-based arrhythmia detection model executes in under 200 milliseconds, and forward an actionable alert to a clinician's tablet application for review and potential intervention.

In some configurations, the platform may be designed for scalability and high availability. Redundant power supplies, RAID-configured storage arrays, load-balancing servers, and failover clusters may be used to maintain uninterrupted operation. Cloud-deployed versions may use auto-scaling groups and container orchestration platforms to dynamically allocate computing resources based on workload demands.

The hardware environment may support multiple functional software modules corresponding to the methods described herein. For example, a clinical decision support engine may generate actionable insights, a clinical data exploration and natural language processing module may perform document processing and visualization, a medication adherence and hospitalization tracker may monitor patient compliance and health outcomes, a quality measure automation engine may support HEDIS workflows, a task triage and workflow manager may optimize provider assignments, a population health analytics module may report on community health trends, and ensemble model processing may integrate multiple AI models into cohesive recommendations. These modules may share a common data layer and unified access control system, enabling cross-referencing between datasets and ensuring consistent security and privacy protections across functions. n certain embodiments, the platform and associated devices may be implemented in compliance with applicable regulatory frameworks, such as HIPAA for patient data privacy, IEC 62304 for medical device software lifecycle management, and FDA Class II medical device integration requirements for connected diagnostic systems.

In certain embodiments, a method for enhancing clinical decision-making and workflow optimization is carried out by one or more processors executing instructions stored in memory. The method begins with collecting data from multiple healthcare sources, including but not limited to electronic health records (EHRs), medical imaging systems, and patient-reported outcomes. The collecting operation may include receiving structured data, such as coded laboratory results and medication lists, as well as unstructured text in the form of clinician notes and narrative reports, and further includes receiving imaging pixel arrays acquired from modalities such as computed tomography, magnetic resonance imaging, or ultrasound. The collection step may be performed via secure network communication interfaces and standardized protocols such as HL7 or DICOM, thereby enabling the processors to assemble a multimodal dataset that incorporates structured, unstructured, and image-based information in a form suitable for downstream analysis.

[symbol]

The collected data is analyzed by the one or more processors using an artificial intelligence (AI) or machine learning (ML) model that is stored in memory and executed in hardware. The model may be a deep neural network, convolutional network, transformer model, or other architecture adapted for healthcare data processing. The analyzing step may include normalizing the multimodal data so that disparate values are placed into a consistent scale or representation. It may further include generating embeddings of unstructured clinical notes using natural language processing techniques that capture the semantic content of patient descriptions, clinician observations, or outcome surveys. The embeddings can then be combined with feature vectors extracted from medical images, for example by segmenting images to identify regions of interest and producing quantitative biomarkers or pattern maps. The fused representation that results from combining structured attributes, unstructured text embeddings, and imaging features allows the ML model to generate predictive outputs that are more robust than those obtainable from a single data source. The predictive outputs may include risk scores indicative of disease progression or adverse events, anomaly detection alerts highlighting deviations from baseline or population norms, and treatment prioritization flags that signal the need for urgent clinical attention.

Following the generation of predictive outputs, the method presents the results by causing a clinician-facing display device to render a unified graphical user interface. The unified interface may be displayed on a workstation monitor, a tablet, or other computing device accessible to healthcare providers. Within this interface, predictive outputs are presented in real time together with context-sensitive fields of the electronic health record, such that a risk score may appear adjacent to a patient's laboratory panel, an anomaly alert may be shown alongside an imaging report, or a treatment prioritization flag may be embedded in a triage task list. By rendering results in this integrated and context-aware fashion, the system reduces redundant user interactions, streamlines clinical workflow, and ensures that the generated insights are provided to healthcare providers through the unified interface without requiring additional navigation or manual data reconciliation.

The disclosed embodiments therefore provide technical improvements to the way multimodal healthcare data are processed and displayed. The system not only harmonizes structured, unstructured, and imaging inputs into a coherent representation, but also executes complex AI models in hardware to produce outputs with reduced latency and increased accuracy. By embedding these outputs directly into EHR interfaces in a context-sensitive manner, the method reduces cognitive load on clinicians and improves the efficiency of healthcare delivery. The combination of multimodal data normalization, embedding generation, imaging feature fusion, and real-time interface integration represents a concrete technological advance over conventional approaches that merely aggregate or display information without addressing the computational challenges inherent in healthcare data interoperability and clinical workflow optimization.

In certain embodiments, a method for exploring and presenting clinical data is performed by one or more processors executing instructions stored in non-transitory computer-readable media. The method begins with receiving a plurality of clinical documents from various healthcare sources, including structured electronic health records (EHRs), unstructured clinician notes, and imaging-derived reports such as radiology summaries. The receiving operation may occur over secure network communication interfaces using standardized data exchange protocols, and results in the assembly of a heterogeneous collection of clinical information stored in memory for downstream processing.

Once received, the clinical documents are processed using natural language processing (NLP) algorithms executed in hardware. The NLP algorithms may include tokenization of text into component terms, parsing of grammatical structures, and generation of embeddings that capture semantic relationships between medical concepts. By embedding and normalizing the textual data, the system extracts relevant information that can be associated with corresponding patient identifiers, clinical events, or time stamps.

The method further includes highlighting selected data points within the processed documents. Such data points may include, by way of example, diagnoses, treatment plans, and medication history. Highlighting may be accomplished by associating extracted tokens with contextual metadata and display attributes, such that relevant terms are visually emphasized when rendered on a display device. This step allows clinicians to immediately identify critical patient information without manually scanning lengthy narrative text.

Following highlighting, the extracted information is organized into an actionable format. In some embodiments, the organizing includes structuring the data into indexed records stored in memory. The indexed records may support cross-referencing between diagnoses, medications, and associated treatment plans, thereby providing an efficient framework for clinical review. Organizing the data in this manner produces a computer-implemented structure that facilitates rapid retrieval and reduces manual data reconciliation tasks.

The method also provides a search function implemented as a software module executed by the one or more processors. The search function enables clinicians to query the indexed records for specific keywords or clinical concepts and returns relevant passages or data points with reduced latency. The search capability may rely on inverted indices, embedding-based similarity search, or other computational techniques that improve retrieval efficiency compared to manual review.

In addition, the method enables tagging and categorization of the clinical data. The tagging operation may include writing metadata values to storage that are associated with indexed records, thereby permitting grouping of related data by diagnosis type, treatment pathway, or other clinically relevant categories. Categorization of this kind improves management of large volumes of records and facilitates interoperability with external healthcare systems.

Finally, the method includes displaying the organized and highlighted data by causing a clinician-facing display device to render a graphical user interface. The interface presents the information in real time, integrating highlighted data points, searchable fields, and tagged categories within a user-friendly layout. By presenting clinical data in this organized and interactive format, the method reduces redundant navigation, lowers clinician cognitive load, and improves overall workflow efficiency in the healthcare environment.

In some embodiments, a method for monitoring medication adherence and hospitalization events is implemented by one or more processors executing instructions stored in non-transitory computer-readable media. The method begins with collecting data from multiple healthcare sources. Such data may include structured pharmacy records indicating prescription fill dates and dosage instructions, timestamped refill transactions obtained through pharmacy information systems, unstructured patient self-reports entered through survey applications or mobile devices, and structured or unstructured electronic health records (EHRs) accessed via hospital servers. The collecting operation may be performed over secure network communication interfaces and may include normalization of input data into a consistent format suitable for downstream analysis.

Once collected, the data are used by the one or more processors to track patient adherence to prescribed medications. In one embodiment, adherence tracking includes cross-referencing prescription fill dates against expected dosing intervals and computing an adherence metric representing the proportion of doses taken as prescribed. The adherence metric may be stored in memory as a time-series data structure, allowing subsequent longitudinal analysis. The system may also record variations in self-reported dosing schedules or skipped medication events provided by the patient.

The method further includes identifying patterns of medication non-adherence. In certain embodiments, the processors execute algorithms that perform time-series analysis, statistical anomaly detection, or pattern recognition across the adherence metric. Through these algorithms, missed doses, irregular intake intervals, or extended gaps between prescription refills can be automatically detected and flagged. By embedding the anomaly detection into the adherence timeline, the system provides a machine-implemented capability to recognize clinically relevant deviations that might otherwise remain hidden.

In addition to adherence analysis, the system monitors hospitalization events. Monitoring includes receiving admission, discharge, and length-of-stay data from hospital information systems or clinical event feeds. The hospitalization events are synchronized with the adherence timeline so that admissions and discharges are placed in temporal alignment with medication adherence patterns. This synchronization produces a unified patient record in memory that associates hospitalization events with contemporaneous adherence behaviors.

The unified patient record is then presented to healthcare providers by causing a clinician-facing display device to render a graphical user interface. The interface may display a timeline integrating adherence metrics with hospitalization events, enabling clinicians to observe correlations between non-adherence and hospitalizations. By embedding adherence data and hospitalization events into a single interactive display, the interface reduces the need for providers to switch between separate pharmacy, EHR, and hospital dashboards, thereby reducing redundant navigation and improving workflow efficiency.

The method also includes alerting healthcare providers to potential adherence issues or recent hospitalizations. In some embodiments, the processors generate automatic alerts triggered when adherence metrics fall below a predetermined threshold or when a new hospitalization event is received from an external system. Alerts may be delivered as pop-up visual indicators within the graphical interface, as color-coded highlights on the timeline, or as electronic notifications routed to a provider's device. The alerting functionality ensures that potential risks are surfaced in real time rather than through retrospective chart review.

Finally, the processors generate reports that summarize medication adherence and hospitalization events for individual patients. Reports may be formatted as structured electronic files, such as PDF documents, HL7-compliant messages, or database records, that are suitable for archival, transmission, or integration with external clinical systems. These reports may include graphical timelines, adherence percentages, lists of missed doses, and summaries of hospital admissions and discharges. By producing structured reports directly from the unified patient record, the system reduces manual data compilation and ensures consistent formatting across patients.

Through this combination of automated data collection, algorithmic analysis, synchronization of adherence and hospitalization data, real-time alerts, and structured report generation, the disclosed method improves the functioning of healthcare computing systems. The method reduces latency in identifying non-adherence, improves interoperability across disparate healthcare sources, and lowers clinician workload by integrating heterogeneous data into a unified, actionable interface. These technical improvements represent a concrete application of computer technology in the domain of healthcare data management and workflow optimization.

In some embodiments, the method of claim 1 may be extended to further include automation of routine administrative tasks. Such automation may be carried out by one or more processors executing stored instructions that access scheduling databases, billing systems, and documentation templates. For example, appointment scheduling may be automatically generated by cross-referencing provider calendars with patient availability stored in memory, billing may be executed by populating structured claims forms from encounter data, and documentation tasks may be completed through template-driven insertion of patient-specific information. By offloading these repetitive tasks to computing processes, provider burden is reduced and clinical workflows are streamlined.

The method may further include providing decision support alerts for potential issues such as drug interactions or contraindications. In one embodiment, the processors execute rule-based and machine learning algorithms that compare prescribed medications against known interaction libraries and patient comorbidities. When a potential conflict is detected, the processors generate an alert object in memory, which is then rendered as a notification or visual highlight within a clinician-facing graphical user interface. These alerts may be integrated directly into electronic health record (EHR) entry screens, thereby enabling real-time decision support during patient encounters.

To ensure interoperability with existing healthcare systems, the method may further include executing standardized data exchange protocols. In some embodiments, the processors initiate communication using HL7 and FHIR-compliant interfaces to import or export structured patient data. Interoperability ensures that recommendations, alerts, and administrative updates generated by the system are seamlessly shared across hospital systems, payer platforms, and third-party health applications. The use of standard protocols further reduces integration latency and improves reliability in data exchange.

In certain embodiments, the AI or machine learning models are continuously updated with newly received data to improve accuracy and relevance. Updating may include writing new training data to memory, fine-tuning stored model parameters, and refreshing knowledge bases to reflect the latest clinical guidelines. Updates may be performed incrementally in real time or as scheduled retraining cycles. The updating process ensures that predictive outputs and alerts remain aligned with current medical evidence and population health trends.

The automated administrative tasks may also include auto-scribing of clinical notes. In some embodiments, the processors execute natural language processing (NLP) algorithms to transcribe spoken words captured during a clinical encounter into structured documentation. The NLP module may further classify the transcribed text into relevant sections of the patient record, such as history of present illness, review of systems, and treatment plan. By automatically generating clinical documentation, the system reduces the manual typing burden on providers and improves data consistency.

In certain embodiments, the decision support alerts may further include predictive analytics for identifying high-risk patients. The processors may execute time-series analysis of laboratory values, vital signs, and adherence records, and apply risk stratification algorithms to compute probability scores for adverse outcomes. Patients exceeding risk thresholds are flagged in memory and surfaced to providers via color-coded indicators within the GUI. This predictive capability enables proactive intervention before adverse events occur.

The method may further include integrating patient data from wearable devices and remote monitoring systems into a unified platform. In one embodiment, the processors receive heart rate, activity level, blood glucose, or oxygen saturation data from wearable sensors via secure wireless protocols. The incoming data are normalized and stored in memory alongside EHR records, thereby enabling longitudinal monitoring and correlation with clinical events. Integration of remote data into the unified platform expands the dataset available for predictive modeling and enhances care continuity.

In some embodiments, the processors provide a dashboard interface rendered on a display device. The dashboard may present real-time visualizations of both patient-level and population-level health metrics. Graphical displays may include adherence timelines, risk stratification pyramids, and population prevalence heatmaps. Interactive elements allow providers to filter, drill down, or expand datasets to tailor the view to clinical needs. By embedding such dashboards within existing clinical systems, the method improves situational awareness and decision-making efficiency.

The collected data may further include genetic sequence information and lifestyle factors. In certain embodiments, genomic data are parsed into encoded attributes such as single-nucleotide polymorphisms and stored in memory for use in pharmacogenomic modeling. Lifestyle factors such as diet, exercise, and social determinants of health may be recorded through patient questionnaires or mobile applications, and converted into structured attributes. By extending the dataset to include genetic and lifestyle information, the system provides a richer foundation for predictive analytics and personalized medicine.

In one embodiment, the processors generate personalized treatment recommendations based on the analyzed data. A trained machine learning model executed in hardware may evaluate genetic, clinical, and lifestyle attributes and output tailored therapy suggestions. Recommendations may be displayed in real time within the GUI and may include ranked treatment options, dosing adjustments, or lifestyle modification prompts. Provider interactions with the recommendations are stored in memory and incorporated into subsequent iterations to refine personalization.

The method may further facilitate collaboration among healthcare providers. Collaboration may be achieved by synchronizing shared access to patient records across networked devices. Updates made by one provider are propagated through memory and reflected across authenticated user sessions to maintain consistency. Secure in-application messaging tools embedded in the GUI allow providers to exchange notes, flag suspect data, or confirm task assignments without leaving the interface. This collaborative functionality supports team-based care models.

In certain embodiments, the method includes implementing security measures to ensure patient data privacy and compliance with regulatory standards. The processors may execute encryption protocols for data at rest and in transit, apply authentication routines such as multi-factor login, and enforce access control rules stored in memory. Compliance modules may monitor usage logs and trigger alerts if regulatory rules are violated. These technical safeguards ensure that sensitive clinical data remain protected while enabling interoperability.

Finally, the method may further include enabling telehealth consultations and remote patient management. In some embodiments, the processors execute audio/video communication protocols integrated into the GUI, permitting real-time interaction between providers and patients. Data exchanged during telehealth sessions, such as remote monitoring values or annotated images, are securely transmitted and stored in memory for integration with patient records. By embedding telehealth capabilities directly into the decision support system, providers are able to extend care delivery beyond traditional settings.

The preceding disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it is understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like) and may be used interchangeably with “one or more.” The phrase “only one” or similar language is used where only one item is intended. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either”or “only one of”).

One or more elements or aspects or steps, or any portion(s) thereof, from one or more of any of the systems and methods described herein, may be combined with one or more elements or aspects or steps, or any portion(s) thereof, from one or more of any of the other systems and methods described herein and combinations thereof, to form one or more additional implementations and/or claims of the present disclosure.

One or more components, steps, features, and/or functions illustrated in the figures may be rearranged and/or combined into a single component, block, feature, or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software and/or embedded in hardware. In certain implementations, the platform may be configured to process data from up to, e.g., 10,000 concurrently connected patient devices, or more, sustaining throughput sufficient to generate alerts with a latency of less than one second in 95% of cases, thereby enabling near real-time clinical responsiveness. Power redundancy may be provided by dual power supplies connected to independent uninterruptible power supply (UPS) systems, ensuring continued operation during primary power failure. Storage subsystems may utilize hot-swappable drives within RAID arrays, permitting replacement of failed disks without downtime. Network paths may include redundant switches and automatic route failover to maintain connectivity during hardware or link failures.

In some embodiments, a method for automating suspect hit processing within Healthcare Effectiveness Data and Information Set (HEDIS) workflows is executed by one or more processors operating in conjunction with memory and non-transitory computer-readable media. The method begins with analyzing patient data records to identify potential quality measures relevant to HEDIS standards. Patient data may include structured claims files, coded entries from electronic health records (EHRs), and unstructured clinical notes. The analyzing step may include parsing structured data fields, tokenizing and embedding unstructured text, and mapping coded entries to measure definitions stored in a HEDIS measure library. In this way, patient-level events and attributes are normalized and aligned with standardized HEDIS categories.

Following analysis, the one or more processors automatically generate suspect hits based on predefined criteria and algorithmic rules stored in memory. These rules may include Boolean logic conditions, numerical threshold comparisons, or pattern-matching operations applied to the patient data. For example, a refill gap greater than a predefined number of days may trigger a suspect hit for a medication adherence measure. The generated suspect hits are stored as data objects in memory, each tagged with references to the originating patient records and measure definitions.

Once generated, suspect hits are prioritized. In certain embodiments, the processors execute a scoring algorithm that assigns weights to each suspect hit based on relevance and potential impact on quality assessments. The scoring may account for the type of measure (e.g., preventive screening vs. chronic condition management), the completeness of the supporting data, and the recency of the underlying event. The prioritized suspect hits are then arranged in an order suitable for presentation to reviewers, with high-impact or time-sensitive hits placed at the top of the list.

The prioritized suspect hits are presented to healthcare providers by causing a clinician-facing display device to render a graphical user interface (GUI). The GUI may display a ranked listing of suspect hits alongside contextual excerpts of patient records that triggered each hit. For example, the GUI may show the relevant medication list, laboratory value, or encounter summary directly adjacent to the suspect hit flag. This integrated presentation reduces redundant navigation between separate data systems and allows reviewers to efficiently confirm or dismiss suspect hits.

When a provider confirms a primary suspect hit, the system automatically deprioritizes related suspect hits stored in memory. Deprioritization may include updating dependency links between hits so that secondary or duplicative hits are suppressed from the active display. For instance, if a colorectal cancer screening measure is confirmed based on colonoscopy data, related suspect hits tied to fecal occult blood testing may be automatically deprioritized, thereby reducing unnecessary review burden.

The method further provides detailed explanations and supporting evidence for each suspect hit. Within the GUI, the system may highlight the specific patient data fields that satisfied the predefined criteria, such as laboratory values exceeding a threshold or procedure codes appearing in the medical record. By exposing the evidentiary basis for each hit, the system improves reviewer trust and reduces manual chart audit requirements.

Finally, the one or more processors generate reports that summarize suspect hit findings and their current status. Reports may include tabular listings of confirmed, pending, and deprioritized hits, along with associated evidence and timestamps. Reports may be output as structured electronic files, such as HL7-compliant messages, XML documents, or database tables, and may be configured for archival, transmission, or integration with external quality management platforms.

Through this sequence of operations, the disclosed method provides concrete technical improvements over manual HEDIS chart review and traditional data processing. By normalizing heterogeneous data, applying algorithmic rules for automatic suspect hit generation, scoring and prioritizing hits with computational efficiency, and dynamically updating dependencies to suppress redundant review, the system enhances the functioning of healthcare computing workflows. Moreover, by integrating evidence directly into a graphical user interface and generating structured reports, the system reduces latency, minimizes provider burden, and improves interoperability with downstream quality management tools. These enhancements represent practical applications of computer technology that improve the efficiency and accuracy of HEDIS workflow execution.

In some embodiments, a method for providing artificial intelligence (AI)-driven treatment recommendations and contraindication identification is performed by one or more processors executing instructions stored in non-transitory computer-readable media. The method begins with analyzing patient data including medical history, genetic information, and current medications. Patient data may be received through network interfaces from electronic health record (EHR) systems, genomic sequencing databases, and pharmacy systems. Because such data sources often use heterogeneous formats, the analyzing step may include normalizing structured and unstructured fields into a consistent schema, generating embeddings of genetic sequences to capture clinically relevant features, and applying an AI model stored in memory and executed in hardware to fuse the normalized inputs.

Based on the fused data representation, the processors generate personalized treatment recommendations. The generation step may involve executing machine learning algorithms such as gradient-boosted models, neural networks, or rule-enhanced AI models that assign weights to treatment options according to patient-specific factors such as age, comorbidities, prior treatment response, and pharmacogenomic profiles. The resulting treatment recommendations are stored in memory along with associated confidence scores or probability estimates.

The method further includes identifying potential contraindications associated with the generated recommendations. The processors may access structured drug-drug interaction databases, dosage guidelines, and contraindication rules stored in knowledge bases. Algorithmic checks may be applied to determine whether the recommended treatment conflicts with a patient's existing medical conditions, prescribed medications, or known allergies. Temporal analysis may be employed to detect dosage overlaps or sequencing conflicts. Contraindications identified through this process are linked in memory to the corresponding recommendations.

The treatment recommendations and contraindications are presented to healthcare providers by causing a clinician-facing display device to render a graphical user interface (GUI). The GUI may display the recommendations in real time within context-sensitive areas of the EHR, such as medication order entry fields or diagnostic panels. By embedding the recommendations directly into existing clinical workflows, the interface reduces redundant navigation between applications and improves provider efficiency.

Within the GUI, the method provides detailed explanations for each recommendation. The explanations may include highlighting of the patient data fields that triggered a recommendation, visualizations of model reasoning paths, or citations to underlying clinical guidelines. These explanations allow providers to evaluate the evidence supporting a recommendation without performing manual data reconciliation.

The system also allows healthcare providers to adjust and refine the treatment recommendations based on their clinical judgment. Interactive elements of the GUI, such as sliders, drop-down selections, or direct text entries, permit providers to modify dosage levels, select alternate therapies, or exclude contraindicated options. Adjustments made through the interface are written to memory and incorporated into subsequent recommendation cycles, enabling adaptive personalization of the AI model outputs.

Finally, the method updates the AI algorithms with new medical research and clinical guidelines. Updating may include fine-tuning stored model parameters using newly ingested training data, refreshing rule-based knowledge bases with updated guideline publications, or adjusting decision thresholds to reflect revised standards of care. Updates may be performed in a manner that maintains compliance with clinical workflow latency requirements, ensuring that model outputs remain responsive during patient encounters.

Through this combination of heterogeneous data fusion, algorithmic recommendation generation, automated contraindication detection, context-sensitive interface rendering, interactive refinement, and continuous model updating, the disclosed method provides specific improvements to healthcare computing systems. The method reduces latency in generating personalized treatment recommendations, improves interoperability between genomic, pharmacy, and EHR systems, and lowers provider burden by embedding recommendations and explanations into a unified interface. By automating contraindication checks and enabling real-time integration of new research, the system enhances both the accuracy and efficiency of clinical decision support. These technical enhancements represent a practical application of computer technology that improves the functioning of healthcare information systems rather than merely automating abstract clinical reasoning.

In some embodiments, a method for optimizing task triage workflows in disease management is performed by one or more processors executing instructions stored in non-transitory computer-readable media. The method begins with categorizing clinical tasks based on urgency and importance. Tasks may be derived from structured fields in electronic health records (EHRs), unstructured clinician notes, or scheduling databases. The categorization step may include parsing coded EHR entries, generating embeddings of natural language descriptions, and applying an AI-based scoring model to assign urgency and importance values. The scores are stored in memory alongside task identifiers to create a structured task dataset suitable for downstream processing.

Once categorized, tasks are prioritized by the one or more processors. Prioritization may include executing a ranking algorithm that elevates time-sensitive tasks such as follow-up appointments, medication refills, or diagnostic tests. In certain embodiments, the algorithm compares urgency scores against time thresholds to dynamically adjust task ranking. The prioritized list is stored in memory and continuously updated as new tasks are created or existing tasks are modified.

The system further allocates tasks to appropriate healthcare providers. Allocation may include retrieving provider availability data from scheduling systems and cross-referencing provider credentials with task requirements. Workload balancing algorithms may be applied to prevent overscheduling and to ensure equitable distribution of tasks among providers. Allocation results are written into the task records stored in memory, with each record updated to include a provider identifier and scheduled time.

To ensure timely task completion, the processors generate alerts and reminders. These alerts may take the form of electronic notifications sent to provider devices or visual indicators presented within a clinician-facing graphical user interface (GUI) rendered on a display device. Alerts are automatically triggered as task deadlines approach, and reminders may be configured to recur until the task is marked as complete in memory.

The GUI also enables healthcare providers to track task status and progress. As providers confirm task completion, reassign tasks, or append notes, the updates are stored in memory and immediately reflected in the GUI. Real-time synchronization between memory and the display device ensures that providers always have access to the most current status of their assigned tasks.

Collaboration among healthcare teams is facilitated by allowing task assignments and updates to be shared across multiple authenticated user sessions. In one embodiment, updates made to a task record in memory are synchronized across networked devices so that all authorized team members view the same version of the record. This shared access ensures consistency of updates and enables teams to coordinate task execution in real time.

Finally, the system analyzes task completion data to identify workflow bottlenecks and areas for improvement. Task records stored in memory may be aggregated statistically to measure average completion times, identify tasks most frequently delayed, and assess provider workload distribution. Latency analysis may be used to pinpoint delays in scheduling or execution, while utilization reports can highlight inefficiencies across departments. The results of this analysis are output as structured files or database entries that may be integrated with external quality management systems for further review.

Through this sequence of categorization, prioritization, allocation, alert generation, progress tracking, collaboration support, and performance analysis, the disclosed method provides concrete technical improvements to healthcare computing workflows. By automating triage and allocation, the system reduces latency in task completion, prevents redundant provider workload, and streamlines communication between care teams. By integrating prioritized task data into a real-time graphical interface and generating structured reports for quality management, the system enhances both efficiency and interoperability. These improvements represent a practical application of computer technology that extends beyond abstract task management concepts, thereby demonstrating a specific enhancement to the functioning of healthcare information systems.

In some embodiments, a method for providing clinical decision support using an ensemble machine learning model framework is carried out by one or more processors executing instructions stored in non-transitory computer-readable media. The method begins with integrating multiple machine learning models, each specialized in a specific aspect of patient care. For example, a first model may be specialized in diagnostic classification using laboratory results, a second model may perform imaging-based analysis to identify radiographic features, and a third model may extract information from unstructured clinical text. The integration step may include storing individual model outputs in memory and applying a model fusion layer that combines the outputs into a unified ensemble representation, thereby enabling joint interpretation of multimodal patient data.

After integration, the processors analyze patient data including clinical records, laboratory results, and imaging data. The analysis step may include normalizing heterogeneous input formats into a consistent schema, extracting feature vectors from medical images, and embedding unstructured clinical notes using natural language processing techniques. The normalized and embedded inputs are then processed by the ensemble framework, which executes the integrated models to generate predictive outputs.

The processors generate comprehensive insights and predictions based on the ensemble framework's outputs. In some embodiments, the insights include risk scores, diagnostic likelihood estimates, and treatment prioritization rankings. Because multiple specialized models contribute to the ensemble, the generated predictions are more comprehensive and accurate than predictions from a single model. The insights are stored in memory and linked to corresponding patient identifiers.

The generated outputs are presented to healthcare providers by causing a clinician-facing display device to render a graphical user interface (GUI). The GUI may display the ensemble predictions in real time, embedded into context-sensitive areas of the electronic health record (EHR). For example, risk scores may appear adjacent to laboratory panels, diagnostic suggestions may be displayed alongside imaging reports, and treatment prioritization recommendations may be surfaced during medication order entry. Embedding predictions directly into the EHR interface reduces redundant navigation and improves clinical workflow efficiency.

The ensemble framework is validated and calibrated to ensure accuracy and consistency of outputs. Validation may include applying the framework to historical patient datasets stored in memory, while calibration may involve adjusting model weighting coefficients or confidence thresholds. In some embodiments, the calibration process equalizes outputs across patient subpopulations, thereby improving fairness and reducing bias in clinical predictions.

The GUI also provides explanations and justifications for the generated insights. Explanations may include highlighting patient data fields that influenced the prediction, displaying the relative contributions of individual submodels, or providing citations to supporting clinical guidelines. This transparency allows healthcare providers to understand the reasoning behind a recommendation and supports trust in the system.

Finally, the processors continuously update the ensemble framework with new data and medical research. Updating may include fine-tuning stored parameters using recent patient data, refreshing calibration datasets with updated standards of care, or retraining one or more submodels with newly available medical research. Updates may be executed in memory with attention to latency thresholds so that predictions remain available in real time during clinical workflow.

Through this combination of model integration, multimodal data normalization, ensemble prediction, calibration, explanation, and continuous updating, the disclosed method provides specific technical improvements to healthcare computing systems. By integrating specialized models into an ensemble framework, the method improves prediction accuracy and consistency compared to individual models. By embedding outputs into a real-time, context-sensitive interface, the system reduces provider navigation burden and improves workflow efficiency. By providing transparent explanations and automatic updating of models with new data, the system enhances both the usability and reliability of clinical decision support. These improvements represent a practical application of computer technology that improves the functioning of healthcare information systems, rather than merely automating abstract clinical reasoning.

In certain embodiments, a method for tracking population health metrics and medication adherence is implemented by one or more processors executing instructions stored in non-transitory computer-readable media. The method begins with collecting data on population health metrics, including chronic disease prevalence, risk factors, and healthcare utilization. The collecting step may include receiving structured claims data, laboratory test results, and aggregated encounter records via secure network communication interfaces. These data are normalized into a consistent schema and stored in memory for downstream analysis.

Once collected, the method continues with monitoring medication adherence by analyzing pharmacy records, patient self-reports, and electronic health records (EHRs). In some embodiments, the processors reconcile prescription fill dates against prescribed dosing schedules, parse unstructured patient notes for references to medication intake, and compute adherence metrics. The adherence metrics may be represented as time-series data objects stored in memory, thereby enabling temporal analysis of medication-taking behavior.

The processors then identify patterns of medication non-adherence. The identification step may include executing anomaly detection algorithms, such as statistical thresholding, clustering, or temporal pattern recognition routines, to detect missed doses, irregular intake intervals, or extended gaps between refills. Detected patterns are flagged and stored in memory, where they may be associated with patient identifiers and linked to clinical risk factors.

The method further includes providing healthcare providers with visualizations and reports on both population health metrics and medication adherence. This is accomplished by causing a clinician-facing display device to render a graphical user interface (GUI). The GUI may include dashboards displaying population-level trend graphs, patient stratification heatmaps, and adherence timelines. In some embodiments, the interface integrates with existing EHR displays so that context-sensitive overlays present adherence and risk information alongside patient charts.

The processors also stratify patients based on calculated risk levels and adherence patterns. Stratification may be performed by executing clustering algorithms, such as k-means or hierarchical clustering, and by applying threshold-based classification rules. Patients are thereby grouped into intervention categories stored in memory, such as high-risk non-adherent, moderate-risk intermittent, or low-risk adherent groups. This stratification supports targeted clinical interventions.

The system generates electronic alerts for healthcare providers regarding patients with critical adherence issues. Alerts may be automatically triggered when adherence metrics fall below a predetermined threshold or when new risk factors are detected. The alerts may be displayed as visual indicators within the GUI or transmitted as electronic notifications to provider devices. Each alert may link directly to the underlying adherence timeline and patient record excerpts.

Finally, the method offers personalized recommendations and interventions to improve medication adherence. In certain embodiments, the processors retrieve context-appropriate strategies from a knowledge base, such as reminders, counseling prompts, or simplified dosing regimens. These strategies are associated with flagged patient records in memory and displayed in the GUI for provider review. Providers may accept, reject, or modify the suggested interventions, and such interactions are stored to refine subsequent recommendations.

Through this sequence of operations, the disclosed method provides technical improvements to healthcare computing systems. By normalizing heterogeneous inputs and computing adherence metrics as structured time-series data, the system enables automated analysis of patient behavior across large populations. By embedding anomaly detection, clustering-based stratification, and automated alerting into a unified platform, the method reduces latency, minimizes manual chart review, and lowers provider workload. By integrating recommendations into context-sensitive interfaces and updating them based on provider feedback, the system improves usability and adaptability. These enhancements represent a practical application of computer technology that improves the functioning of healthcare information systems, rather than merely automating abstract clinical reasoning.

Reference in the specification to “one embodiment” or “an embodiment” means that a particular feature, structure, or characteristic described in connection with the embodiment is included in at least one embodiment. The appearances of the phrase “in one embodiment” in various places in the specification do not necessarily refer to the same embodiment.

Some portions of the detailed description are presented in terms of algorithms and symbolic representations of operations on data bits within a computer memory. These algorithmic descriptions and representations are the methods used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared or otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following disclosure, it is appreciated that throughout the disclosure terms such as “processing,” “computing,” “calculating,” “determining,” “displaying” or the like, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. It will be appreciated that a variety of programming languages may be used to implement the teachings as described herein.

The figures and the description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this Application. As will be understood by those familiar with the art, the present invention may be embodied in other specific forms without departing from the spirit or essential characteristics thereof. Likewise, the particular naming and division of the modules, routines, features, attributes, methodologies and other aspects are not mandatory or significant, and the mechanisms that implement the present invention or its features may have different names, divisions and/or formats.

Furthermore, as will be apparent to one of ordinary skill in the relevant art, the modules, routines, features, attributes, methodologies and other aspects of the present invention can be implemented as software, hardware, firmware or any combination of the three. Also, wherever a component, an example of which is a module, of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming.

Additionally, the present invention is in no way limited to implementation in any specific programming language, or for any specific operating system or environment. Accordingly, the disclosure of the present invention is intended to be illustrative, but not limiting, of the scope of the present invention, which is set forth in the following claims.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

1. A method for enhancing clinical decision-making and workflow optimization, the method comprising:
collecting, by one or more processors executing instructions stored in memory, data from multiple healthcare sources, including electronic health records (EHRs), medical imaging, and patient-reported outcomes, wherein the collecting includes receiving structured data, unstructured text, and imaging pixel arrays via network communication interfaces;
analyzing, by the one or more processors, the collected data using artificial intelligence (AI) or a machine learning (ML) model executed in hardware, the analyzing including at least normalizing the multimodal data, generating embeddings of unstructured clinical notes, and combining the embeddings with imaging-derived feature vectors to produce predictive outputs that indicate risk levels, anomaly detection alerts, or treatment prioritization flags; and
presenting, by causing a clinician-facing display device to render a unified graphical user interface, the predictive outputs in real time together with context-sensitive fields of the electronic health record, thereby reducing redundant user interactions and streamlining clinical workflow, and delivering the generated insights to healthcare providers through a unified interface.

2. The method of claim 1, further comprising:
automating, by the one or more processors, routine administrative tasks including appointment scheduling, billing, and documentation;
providing, by the one or more processors, decision support alerts for potential issues including drug interactions or contraindications;
executing standardized data exchange protocols, including HL7 and FHIR, to ensure interoperability with existing healthcare systems and standards; and
continuously updating, by the one or more processors, the AI or ML models with newly received data to improve accuracy and relevance.

3. The method of claim 2, wherein the automated routine tasks include autoscribing of clinical notes using natural language processing algorithms executed by the processors.

4. The method of claim 1, wherein the decision support alerts include predictive analytics generated by executing time-series analysis and risk stratification algorithms to identify high-risk patients.

5. The method of claim 1, further comprising integrating, by the one or more processors, patient data from wearable devices and remote monitoring systems into a unified platform stored in memory.

6. The method of claim 1, further comprising providing, by causing a display device to render a dashboard interface, real-time data and visualizations of patient and population health metrics.

7. The method of claim 1, wherein the collected data further includes genetic sequence information and lifestyle factors encoded as structured attributes in memory.

8. The method of claim 1, further comprising generating, by the one or more processors, personalized treatment recommendations using a trained machine learning model executed in hardware.

9. The method of claim 1, further comprising facilitating collaboration among healthcare providers by synchronizing shared access to patient data across networked devices and providing secure communication tools through the graphical user interface.

10. The method of claim 1, further comprising implementing, by the one or more processors, encryption protocols, authentication routines, and access controls to ensure patient data privacy and compliance with regulatory standards.

11. The method of claim 1, further comprising enabling telehealth consultations and remote patient management by executing integrated audio/video communication protocols and secure data exchange between provider and patient devices.

12. A system for enhancing clinical decision-making and workflow optimization, the system comprising:
one or more processors; and
one or more non-transitory computer-readable media storing instructions that, when executed by the one or more processors, cause the one or more processors to:
collect, via network communication interfaces, data from multiple healthcare sources including electronic health records (EHRs), medical imaging systems, and patient-reported outcomes, wherein the collecting includes receiving structured data, unstructured text, and imaging pixel arrays;
analyze the collected data using an artificial intelligence (AI) or machine learning (ML) model executed in hardware, the analyzing including at least normalizing the multimodal data, generating embeddings of unstructured clinical notes, and combining the embeddings with imaging-derived feature vectors to produce predictive outputs that indicate risk levels, anomaly detection alerts, or treatment prioritization flags; and
present the predictive outputs by causing a clinician-facing display device to render a unified graphical user interface in real time together with context-sensitive fields of the electronic health record, thereby reducing redundant user interactions, streamlining clinical workflow, and providing the generated insights to healthcare providers through the unified interface.

13. The system of claim 12, wherein the instructions, when executed, further cause the one or more processors to:
automate routine administrative tasks, including appointment scheduling, billing, and documentation;
provide decision support alerts for potential issues, such as drug interactions or contraindications;
ensure interoperability with existing healthcare systems and standards; and
continuously update the AI and ML models with new data to improve accuracy and relevance.

14. The system of claim 13, wherein the automated routine tasks include autoscribing of clinical notes.

15. The system of claim 12, wherein the decision support alerts include predictive analytics for identifying high-risk patients.

16. The system of claim 12, wherein the instructions, when executed, further cause the one or more processors to integrate patient data from wearable devices and remote monitoring systems into a unified platform.

17. The system of claim 12, wherein the instructions, when executed, further cause the one or more processors to provide a dashboard interface that displays real-time data and visualizations of patient and population health metrics.

18. The system of claim 12, wherein the collected data includes genetic information and lifestyle factors.

19. The system of claim 12, wherein the instructions, when executed, further cause the one or more processors to generate personalized treatment recommendations based on the analyzed data.

20. The system of claim 12, wherein the instructions, when executed, further cause the one or more processors to facilitate collaboration among healthcare providers through shared access to patient data and communication tools.

21-99. (canceled)
